(19) United States
(12) Patent Application Publication
(10) Pub. No.: US 2017/0312727 A1
(43) Pub. Date: Nov. 2, 2017

(54) ANALYSIS METHOD ON THE BASIS OF AN ARRAY

(71) Applicant: ALBERT-LUDWIGS-UNIVERSITAET FREIBURG, Freiburg (DE)

(72) Inventor: Guenter Roth, Freiburg (DE)

(73) Assignee: ALBERT-LUDWIGS-UNIVERSITAET FREIBURG, Freiburg (DE)

(21) Appl. No.: 14/407,073

(22) PCT Filed: Jun. 14, 2013
(86) PCT No.: PCT/EP2013/062373

§ 371 Date: Dec. 11, 2014

(30) Foreign Application Priority Data
Jun. 14, 2012 (DE) 10 2012 105 165.8
Feb. 11, 2013 (EP) 13154826.5

(51) Int. Cl. B01J 19/00; C12Q 1/68; G01N 33/543

(52) U.S. Cl. CPC B01J 19/0046; G01N 33/543; C12Q 1/6837; B01J 2219/00317; B01J 2219/00626; B01J 2219/00596; B01J 2219/00585; B01J 2219/005; B01J 2219/00722; B01J 2219/00725; B01J 2219/00702

(57) ABSTRACT

The invention relates to a method for analyzing molecular properties and/or reaction conditions, comprising a step of providing a first store having a first surface, wherein a specific selection of sample molecules is directly or indirectly bonded to the surface in a defined arrangement, a step of producing at least two transfer stores, wherein at least two additional surfaces are provided, and a reaction step, selected from the group comprising a transfer reaction, an amplification reaction, and/or a derivatization reaction, whereby product molecules can arise and said product molecules and/or the sample molecules bond to the surfaces, wherein there is a clear spatial association between the sample molecules of the first store and the product molecules and/or sample molecules of the transfer stores and the first store, the transfer stores, the sample molecules, the product molecules, the transfer reaction, the amplification reaction, and/or the derivatization reaction is analyzed.

The optimization of biochemical processes, the discovery of a molecule having desired properties or the derivation of a modified molecule having improved properties are often implemented using an approach known as “screening.” Screening refers to the very extensive, more or less systematic testing of a number of reaction conditions with a number of reactants and interaction partners. Screening also includes improving the biochemical process management, for example, the concentration of the enzyme, the substrate, the selection of cofactors, their concentration, adjusting the pH level, etc. Optimization of the properties of molecules by minor modification is frequently also referred to as “scanning” or “lead optimization.”

Almost all screening methods begin with an extensive pool of various molecules. These pools are often referred to as a “molecular library” and may contain up to 10¹⁵ or more different molecules. With such a large number, it is understandable that it is impossible to test each molecule individually but instead testing is carried out by means of the pool, yielding increasingly restrictive results until a manageable number usually a few tens to thousands of molecules remain. These candidates mostly have the desired properties because of the preceding selection. This can then be validated in individual tests, and a ranking list can be compiled, indicating how well the individual molecules correspond to the desired properties. The candidates listed most often are then investigated in detail and are optimized further or used directly if the results of this testing are successful.

On the whole, screening methods are very time-consuming, labor-intensive and cost-intensive. Although they begin with up to 10¹⁵ molecules, frequently only a few dozen to hundreds selected molecules (usually those with the highest listing) are ultimately tested in detail because of the extensive follow-up testing of the candidates. Since many of the selection methods have a preferential or statistical selection bias for other secondary properties in addition to the desired molecular properties, it often occurs that even interesting molecules having the desired properties (but without the secondary properties) are sacrificed to this bias and are therefore no longer included in the final pool.

This bias becomes even greater, the more often a selection is carried out or when a selection condition is set more strongly, because this property and also the secondary property is even more strongly relevant for the persistence of the molecule. Selection with a weak selection pressure in turn allows a great many molecules to advance further, but that in turn means that even more candidates must be investigated.

It would be desirable in general if ultimately more candidates could be investigated at the same time and in particular by simple methods, in particular when 10⁴ to 10⁶ molecules remain in the final pool as a result of weak selection.

After one or more molecules having the desired properties have been identified, the molecular structure may be varied randomly or in a targeted manner in one or more positions. This newly created mutation library is then selected again according to the molecule having the best correspondence with the desired properties. Here again, the results are subject to selection bias and the low number of candidates ultimately analyzed.

For a better understanding of the dependence of properties on the changes in the molecule here, so-called scans are carried out frequently.

One position of the molecule is varied in a targeted manner in this step, and the changes in properties are then measured. This is used with DNA, RNA and proteins in particular. The so-called “alanine scan” is particularly well known for proteins. In this process, each amino acid position on the protein is replaced by alanine. If there is a substitution, usually with an inactive alanine, in this biochemically important location, then the protein no longer has any activity. Important positions can be detected in this way, in particular on enzymes. However, in addition to alanine, there are also 19 other natural amino acids. If one wants to alter two amino acid positions, it is first necessary to create 400 different proteins; for a change in n positions, 20ⁿ different variants of a protein are obtained for each of the 20 amino acids. For even five amino acid positions, this means 3.2 million different proteins. Neither individual synthesis nor measurement is possible with such a large number. Therefore, in most cases, one will make do with a simple alanine scan and then will attempt to derive an optimized molecular sequence and molecular structure from the scan.

It would also be desirable here to ultimately be able to investigate more candidates at the same time and by a simple procedure. In the case of mutation libraries having 10⁴ to 10⁶ mutants in particular, it is of great interest to analyze all the mutants because this then makes the alanine scan superfluous, on the one hand, while supplying a comprehensive data record, on the other hand, because all possible variants have been investigated. This would allow broader use of a subsequent simulation or a systematic analysis of active groups.

In summary, this screening can be divided into three major ranges:
1. Targeted optimization of a biochemical process or a molecular interaction by adaptation of ambient conditions such as salt content, temperature, pH, additives, coenzymes, etc.
2. Discovery of a molecule by selection from a molecular library containing up to 10¹⁵ individuals, based on desired properties that are preferred given preference through selection.
3. Optimization of a molecule or a basic molecular structure by selection from a molecular library, based on desired properties, which are given preference through selection or by means of a systematic investigation (scans) of variants of the basic molecular structure.

Screening methods are of very great interest for the field of pharmacy in particular, which is why there are a number of techniques in this field for synthesizing and analyzing the largest possible number of molecules.

Three main strategies can be differentiated in the prior art.

In so-called individual synthesis, each substance is synthesized and measured individually. This approach has the advantage that one knows from the beginning which molecule is involved and the exact molecular structure is known. The great disadvantage may be seen in the fact that it is technically almost impossible and is also extremely expensive to create or measure more than 10⁶ different molecules.

In so-called combinatorial and/or randomized synthesis, the substances are not synthesized individually but rather in mixtures. This has the advantage that millions of substances can be synthesized in a highly parallel process. However, one disadvantage is that it creates a mixture of molecules, so it is not known which molecule has generated a certain signal. It is subsequently necessary to elucidate the structure of the molecule thereby discovered.

Combinatorial synthesis and replication and/or display methods involve a special biochemical process management. First, a library of substances is created on the basis of DNA or RNA by means of combinatorial synthesis. This DNA or RNA is then packaged in or added onto biologically active “components” (for example, phages, E. coli, yeast cells, ribosomes, etc.). These molecules can then be replicated and thus amplified by activating a replication impulse. The amplification may be carried out in a biologically natural manner, for example, by growth or infection of bacteria and yeasts, or by artificial systems, e.g., DNA polymerase, RNA polymerase, enzyme systems. The advantage of these substance libraries is that they are synthesized only once and can then be “cultured” further. However, these methods are limited to DNA, RNA and proteins in most cases because synthetic active ingredients such as DNA, RNA or proteins are not accessible to biosynthesis to this extent.

If one desires a large number of molecules, then synthesis in microtiter plates often has a low capacity and is therefore eliminated as the agent of choice. Microarrays have proven successful for individual synthesis of 10⁴ molecules or more. It is possible here to create up to 10⁶ different DNA strands by using printing techniques or lithography. These are thus state-of-the-art microarrays for individual synthesis of molecules. However, these systems have only a limited synthesis efficiency (approx. 99% per base for DNA, approx. 96-98.5% per amino acid for proteins), which results in high levels of impurities being expected with long or complex molecules, and/or the maximum length of the molecules that can be synthesized being limited by these impurities.

Combinatorial solid-phase synthesis has proven successful for the state-of-the-art combinatorial synthesis method, in which the molecules are bound to particles. The process known as “split and mix” or “split and recombine” ensures, through the reaction management, that precisely one single species of molecule is constructed on each particle. One disadvantage is that random splits make it impossible to detect a priori on which particle which substance has been constructed.

Three different strategies are then optionally used below.

In the first strategy, the particles are separated from one another and the molecules are each split off separately from the particle. This yields an ultrapure solution of the molecule, parts of which can then be analyzed in the traditional way using a microtiter plate, for example. If the molecule has the desired properties, the ultrapure solution is then analyzed, and the structure of the molecule is thereby elucidated. There have been attempts to label particles for synthesis, so that the path of each individual particle can be followed during the synthesis. However, these methods require a system for labeling the bead and monitoring the location of each bead between the distribution steps. For reasons of synthesis efficiency, it is nevertheless necessary to check once again on whether the particle thus created has also been processed correctly. However, these methods have the advantage that in the case of measurements on 10⁴ components or more, it is possible to infer functional structures on the basis of chemical similarities because it may be assumed that the synthesis is correct in the majority of cases.

With the second strategy, all particles are subjected to a measurement and the signal generation is designed so that the particles with the molecules having the desired property can be sorted out. Then these particles are isolated individually, the molecules are split off and the ultrapure solution is then analyzed. Here again, the goal is to develop methods that will record the individual particles during their synthesis pathway and thus allow the structure to be determined without a subsequent analysis.

The third strategy is to split off all the molecules from all the particles and thus create a mixture containing up to 10¹⁵ different molecules in some cases. These mixtures may also be created by means of other reaction processes, in which the solid phase then does not carry just one species of molecule of a “pure species” but instead has a mixture of molecules. The mixture thereby created is then subjected to selection, i.e., to a process management, which ensures that molecules having the desired property are enriched and molecules having unwanted properties are depleted. Multiple selection processes may be carried out in succession, if necessary, so that there is progressive enrichment of the desired molecules. If the enrichment is great enough, then detection and identification of the molecules may be carried out. Frequently, however, direct identification of the molecules is impossible. Only in the case of DNA, RNA and, in special cases, proteins as well is it also possible to include an amplification step, which allows further enrichment up to the stage of identification.

With the current state-of-the-art methods, it is impossible to characterize a large number of molecules, e.g., 10² to 10⁶ or more molecules, in a highly parallel and simple manner, so that they are linked with regard to their molecular structure as well as their properties. In most cases, a selection is made first and then the structure of individual molecules from the final pool is elucidated, because it is assumed that the best molecules should occur most frequently in the pool. To completely exploit the pool, it is therefore necessary to analyze all the molecules of the pool and to characterize them as directly as possible with regard to their properties.

In addition, with the state-of-the-art methods, it is necessary to perform more than one selection step in order to create a final pool of molecules. In the case of the display methods, these may involve three to five repetitions (phage display) up to 10 to 20 repetitions (SELEX). This is time-consuming and cost-intensive, but the main problem lies in the selection bias, which ensures that even optimal molecules are suppressed because of unsuitable secondary properties, such as poor amplifiability in SELEX, for example, even if these molecules have the best properties otherwise.

The object of the present invention is therefore to provide a method that will make it possible to process a large pool of 10² to 10⁶ or more molecules at the same time, and, in doing so, to analyze their structure as well as properties, so that they are linked together.

### DESCRIPTION OF THE INVENTION

This object is achieved by the independent claims. Advantageous embodiments are characterized in the dependent claims.

In a first preferred embodiment, the invention relates to a method for analyzing molecular properties and/or reaction conditions, comprising the following steps:
a) Supplying a first storage comprising a first surface, wherein
a selection of sample molecules is bound directly or indirectly to the surface in a defined arrangement,
b) Synthesis of at least two transfer storages, comprising:
supplying at least two additional surfaces, and
a reaction step selected from the group of transfer reaction, amplification reaction and/or derivatization reaction, so that the product molecules can be formed, and these product molecules and/or the sample molecules bind to surfaces, such that there is a clear-cut spatial association between the sample molecules of the first storage and the product molecules and/or sample molecules of the transfer storage,
c) Analysis step, comprising analysis of the first storage, the transfer storage, the sample molecules, the product molecules, the transfer reaction, the amplification reaction and/or the derivatization reaction.

In the method described here, an original is created in a first storage with a pool of sample molecules selected in a targeted manner. A first storage may therefore also be referred to as an original in the sense of the invention. This storage has a spatially fixed arrangement of the sample molecules. Each position on the original is clearly linked to one or more sample molecules. Then at least two, preferably a plurality of transfer storages are synthesized on the basis of this original. Various “copies” are possible here. In other words, the transfer storages thus produced may differ from one another. Next, the first storage as well as the synthesized transfer storage and/or copy process itself may be analyzed. This method according to the invention therefore offers a variety of copy and analysis options for what can be used for numerous applications and questions.

It is preferable in particular that the selection (ii) of sample molecules is made by targeted selection. Thus, a higher throughput can be achieved through a skillful combination of sample molecules than is possible in the state-of-the-art method. The selection of sample molecules may be made in a variety of ways. First, it is possible to perform a targeted selection from a large pool of sample molecules. Secondly, however, it is also within the scope of the invention that a pool of molecules created by mutations, starting from a small pool of molecules, can be investigated with the method according to the invention. This may be accomplished first of all by mutation or by permutations of individual molecules up to several molecules. It is not customary in the prior art for a selection to be carried out before the microarray, which is why it was not to be expected that the advantages according to the invention could be achieved through this step, among others. It is also preferable that the sample molecules are selected from a mutation library. This is preferably a molecular library but all the molecules are derived from a starting molecule. In other words, all the molecules of the library are almost identical to the starting molecule and are varied only in defined positions. The total number of variations is the product of the variations per position being varied. Thus if a DNA strand with a length of 100 bases is varied in only 20 positions, and four different bases are inserted at each of the varied positions, then there are 4²⁰ variants. Two molecules of this DNA mutation library will thus correspond to one another in at least 80 DNA bases.

A copy is also understood in the sense of the present invention to be an amplificate of a sample molecule, a derivative of a sample molecule or a transferred sample molecule. A copy in the sense of the invention refers in particular to a product molecule and/or the totality of product molecules. Thus a copy refers not only to identical molecules but also any type of product molecule that may be formed through iv). A copy may therefore also be an amplificate or a derivative. Thus, the copy of a DNA original molecule may be, for example, a DNA product molecule or an RNA product molecule or a protein product molecule.

It is preferred in particular that the reaction step takes place in a cell-free reaction system, especially preferably in a cell-free expression system.

An amplificate of a molecule is preferably formed by amplification of an original molecule. The amplificate may be identical to the original molecule or may be derived from that molecule in a clear-cut way (for example, when the corresponding cDNA is created from DNA).

A derivative of a molecule is preferably the molecule(s) which is/are formed when an original molecule is converted or when amplificates or a molecule have been created and these have been converted or when molecules derived directly or indirectly from the original molecule are created (for example, DNA, which has first been amplified by PCR and then RNA or protein is created from it).

The present invention thus represents a unique amplification as a novel combination of selection, microarray copy technique, screening and process management of individual molecules and/or particles, so that it is possible to spatially separate a pool of sample molecules and particles in a highly parallel manner, to prepare multiple copies of the molecules from the separation pattern and to ensure, on the basis of these copies and/or the copy process itself, that
a) the molecules are present as a pure species (any contaminants that might be present are also detected in the analysis process),
b) the molecules can be copied at least once, preferably repeatedly onto another surface,
c) the molecules can be analyzed and identified with regard to their molecular structure by analyzing the original or one of the copies, and/or
d) the properties of the molecules are determined during the copy process or by analysis of the copy or of the original.

One advantage of the invention lies in the copy step. This step supplies a particularly good result because the transfer storage, preferably “copied microarrays,” is/are supplied in a high quality. This means that, among other things, an unexpectedly high purity can be achieved. Furthermore, the process itself is surprisingly fast. Copying of arrays is not yet established in the state of the art because this technique is associated with difficulties, besides being very time-consuming and expensive in particular. Through the present invention for the first time a method with which these obstacles can be overcome is made available, so that copying of an array can now be used for many different analysis methods, preferably simultaneous analysis methods.

This system has the potential to detect and analyze different sample molecules over several orders of magnitude, of 10² to 10⁶ and more. Additional advantages of this method include the high purity in which the product molecules are obtained after the reaction step. Furthermore, smaller volumes can be used, which contributes toward savings in terms of the reaction components and thus ultimately also savings in the total cost. All these advantages thus yield not only a cost reduction but also a reduction in the labor involved because the reliability of this method is particularly high and therefore tests need not be repeated.

The flexibility and versatility of the method are especially advantageous. There are numerous embodiments and fields of application for the method according to the invention, so that many different questions can be handled by using this method.

The core steps of the method may also be summarized as
Selection of the sample molecules
Creation of the original
Creation of the copy (copies)
Performing the analysis (analyses).

It is a special advantage of the invention that it is possible through the method according to the invention to use especially long sample molecules such as nucleic acid segments, for example, genomic DNA, as sample molecules. This is an enormous advantage in comparison with the prior art. Thus it is stated, for example, in an article by Monya Baker about microarrays (“Microarrays, Megasynthesis,” Nature Methods, 2011, vol. 8, pp. 457) that regardless of the use of an oligonucleotide library, scientists always strive to use or investigate longer and longer oligonucleotides with a lower error rate (“No matter how researchers intend to use libraries of oligonucleotides, they usually want more oligonucleotides, longer oligonucleotides and lower error rates.”). This article also quotes Jay Shendure, a scientist who states that if the possible length were 300 base pairs or even 1000 base pairs, there would be several possibilities that could not be implemented at the present time (“If [the achievable length] was 300 base pairs or even a kilobase, there are a lot of things one could do that one can't do now.”). Precisely this problem can be solved through the present invention. Thus, for example, initial storages and transfer storages can also be produced without any loss of quality and with significantly fewer problems when using DNA lengths of up to 1500 base pairs. This illustrates the point that the invention contributes toward solving a problem for which the technical world was unable to find a satisfactory solution as of the priority date. There was thus an urgent need to find a suitable solution for this problem.

Various microarrays or surfaces resembling microarrays may serve as the “first storages” and a plurality of copies, some of them of different types, can be created and analyzed by various methods. The various embodiments of the individual steps can then be combined freely and thus allow a plurality of applications and/or copies to be produced. The different embodiments of the steps are described below.

In the creation of the first storage—also referred to as the original in the sense of the invention—it is possible to use a molecular library, a pool derived therefrom or a mixture of molecules. The molecules may be freely present in solution or may also be bound to particles or surfaces. Or the molecular species may be separated from one another individually and then added to the original. The molecules may be present individually in solution or two or more species of molecules may be linked to one another. If at least two species of molecules are linked to one another, then it is possible to infer the presence and structure of a second species of molecule from the presence and/or analysis of one species of molecule (molecular tagging).

A molecular library is preferably a mixture of up to 10¹⁵ or more different molecules created by combinatorial chemistry, for example. In most cases, the molecules of a library have similar basic structures or structural patterns which have been randomly combined with one another. The number of possible molecules is calculated as the product of the individual possible variations. For example, one DNA strand may contain four natural building blocks per position. This means that a combinatorial library of a DNA strand 100 bases long already contains 4¹⁰⁰ different DNA molecules.

It is also preferable for the sample molecules to be bound to particles. Due to this embodiment, there can be a better and in particular a targeted “loading” of the first storage.

To now create the first storage, the sample molecules or particles are separated from one another by means of suitable restrictions and applied to the surface of the storage with spatial resolution. The sample molecules are then attached in such a way that they are subsequently unable to easily leave their position. The original is thus a molecular storage with spatial resolution.

In the case of the particle storage, the storage is created by adding particle to the surface. Each particle carries exactly one or more species of molecules. The carrier surface may be structured and the particles may be positioned in various ways on the structuring. If there are at least two species of molecules on one particle, then the presence and structure of the second species of molecule can be inferred from the presence and/or analysis of the one species of molecule (molecular tagging).

It is also preferable that the first storage is a particle transfer storage. In the case of such a storage, the particles are added to the surface. Each particle then carries one or more species of molecules. The carrier surface may be structured and the particles may be positioned in different ways on the structuring. At least one species of molecule is released from the particle, or derivatives or amplificates of at least one species of molecule of the particle are created and these are then transferred to the surface of the transfer storage. Thus the transfer storage constitutes a type of self-copy because some molecules of the particles are copied to the storage. It is preferable if the position information on the sample molecules relative to the particle is retained, i.e., the position of the particle and the position of the sample molecules may be associated with one another. The particle may optionally be removed or used for a subsequent molecular transfer or for creating amplificates or derivatives of molecules.

It is also preferable that the first storage is a molecular storage. With the molecular storage as the original, molecules are optionally added to the surface individually, in coupled form or as mixture. The carrier surface may be structured, may offer preferred positions for attachment of the molecules and the attachment to the structuring may be positioning in different ways. The molecules initially remain in the molecular storage. It is also possible by means of amplification and/or derivatization for the molecular storages to be loaded with amplificates or derivatives of the molecule(s), which are initially contained in the first storage. The molecular storage thus represents a self-copy of the original molecules, so to speak, and thus allows a signal gain. The position information on the molecules is preserved.

It is also preferable that the first storage is a property storage. In the case of a property storage as an original, preferably identical molecules or particles are attached to the surface. The carrier surface may be structured and the attachment to the structuring may be positioned in various ways. The property storage may have different properties inherently or based on an external influence in each position of the storage. This may lead to different properties because of the included microfluidics, microelectronics, the surfaces (structure, coating, material, etc.) or addition of molecules or particles or a combination of these possibilities. These properties may comprise differences of various types (physical, chemical, biochemical) and may include, for example, different volumes, surfaces, wettability, pH, salt contents, biochemical ingredients, electrical charges, electrical, magnetic or dielectric properties, osmotic pressures or additives. The storage of properties is preferably used for optimization of a biochemical or chemical reaction and should implement different reaction conditions. In the usual case, all the positions of the storage of a storage of properties are loaded with identical molecules.

Those skilled in the art are capable of selecting a suitable first storage without having to make an inventive contribution of their own.

All the first storages mentioned above may include a mechanism which makes it possible to release molecules in one position within the storage in a targeted manner. This may be accomplished by releasing the molecules by means of chemical, electrochemical, photochemical or purely electrical/magnetic, thermal mechanisms. In the case of particle storages, the complete particle or parts thereof may be released. The molecules thereby obtained are then ready for any further investigation or modification. A copy may optionally also be prepared from the storage and the molecules may be released from the copy in a targeted manner.

The following mechanisms are preferred here:
Spatially resolved addition of an acid or base, causing a molecular rearrangement that releases molecules;
Spatially resolved creation of acid or base by means of light or electricity, by electrolysis or photolysis;
Cleavage of a chemical group by creating charges locally by means of electricity or light;
Rearrangement of a chemical group by input of light;
Releasing electrostatically bound molecules on charged surfaces by a local change in the electrical field or in the redox potential by means of electricity or light;
Rearrangement of a chemical group by local heating or cooling;
Rearrangement of the chemical groups based on additives or a combination of the aforementioned effects;
Decomposition of a particle and thus release of a the molecules by means of heat, melting, cutting by laser light or triggering a decay process by means of light, chemistry or electromagnetic effects;
Creating physical forces on a particle by means of electrical, magnetic, electromagnetic or dielectric fields or by targeted overheating of liquid in the vicinity of the particle in order to create a force through the resulting expansion;
Mechanical ablation of the surface to which the molecules are applied or mechanical gripping of the particle to remove it from the original.

In the creation of the copy, various reactions may be utilized to create a molecular copy of the original. In all the embodiments described below for creating a copy, the preferred embodiment of the first storage is represented in the form of a storage having cavities. The copy is created for other storages in a similar manner.

To create a copy in the sense of the present invention, the sample molecules may optionally be released from the storage and transferred or the molecules contained therein may be amplified and transferred or amplified and derivatized and certain created derivatives or amplificates may be transferred to the copy. The derivatives may be identical to the original molecules or they may be derived therefrom in a direct or indirect form and can therefore be assigned clearly to the original molecule. For example, first an identical DNA may be created from a DNA and then a base thereof may be exchanged in a certain sequence position by means of an enzyme system, and then this modified DNA may be derivatized to yield RNA or even protein. Since the original sequence of the DNA is known, the sequence of the modified DNA is also known and therefore that of the resulting DNA and/or protein is also known. A copy thus has the following properties:
There is a unique spatial association between the original and the copy, so that, based on knowledge of the location on the copy, it is possible to assign the location to the original, and a location on the original may be associated with a location on the copy in an clear-cut manner.
Clear-cut molecular relationships can be assigned, so that it is possible to ascertain, from analysis of a molecule on one of the copies or on the original, which molecule is involved on each of the copies and the original.

In addition, the surface of a copy may be planar or structured and may itself in turn be an original of which additional copies can be created. The following transfer techniques for the originals are conceivable.

It is preferable that the transfer storage is formed by a transfer copy. In a transfer copy, the molecules of the first storage are transferred directly to the surface of the transfer storage. This means that the molecules are released from the surface of the original, transferred and bound to the copy.

It is also preferable that the transfer storage is formed by a derivatization copy. In the derivatization copy, the sample molecules of the original are derivatized and these derivatives are then transferred to the copy. Derivatives may represent, for example, conversions of the original molecules, which is why depletion takes place here until no more derivatives can be produced because then all the molecules would be consumed.

It is also preferable that the transfer storage is formed by a self-created copy. In the self-created copy, the molecules of the original have a catalytic, enzymatic and/or chemical activity, which ensures that the added molecules are amplified and/or derivatized. These self-created molecules are then optionally transferred to the copy directly or by means of another derivatization or amplification.

It is also preferable that the transfer storage is formed by a combination copy. The combination copy is a parallel or serial linkage of derivatization, amplification or self-creation to create the copy. At least two processes are linked, and may optionally include amplification or derivatization or self-creation. In the preferred case, amplification takes place first because the original molecules are retained here, and then derivatization of the amplificates is carried out or there is further amplification of the amplificates. These may then be further derivatized and/or amplified subsequently in order to create the desired molecules. In the case of an initial derivatization and subsequent amplification, the original is gradually consumed. However, this consumption then takes place much more slowly than is the case with the plain derivatization copy and, in contrast with the former, allows the creation of more copies before the original has been consumed.

In principle, any number of amplification, derivatization and self-creation steps may be linked together before a copy is created.

It is also preferable that the transfer storage is formed by a multi-molecular copy. With the multi-molecular copy, at least two species of molecules are copied from one position. Then at least one of the aforementioned copy creations (direct transfer, amplification, derivatization, self-creation, combination) is used or combined for each species of molecule.

It is also preferable that the transfer storage is formed by a liquid copy. The liquid copy is applied to the first storage as a realizable molecule that is derivatized or amplified by or in the presence of suitable molecules on the original itself. In other words, a spatial association is formed between the creation of the derivatives and/or amplificates and the underlying molecules. These derivatives and amplificates of the added molecule need not necessarily be transferred to a copy. In this preferred embodiment, the statement that the molecules of the original have the generating property for derivatives and amplificates have the generating property for other molecules. This case occurs when there are different enzymes on the original, for example.

It is also preferable that the transfer storage is formed by a DNA-to-DNA copy. The DNA-to-DNA copy corresponds to an amplification copy. In this case, DNA in the original is amplified again to DNA by means of a DNA polymerase. The resulting amplificates may then be bound directly to the copy or may be amplified further by means of a solid phase polymerase reaction on the surface.

In the first experiments, DNA molecules were selected as the sample molecules. A protein “copy” was to be created by the reaction steps. It has surprisingly been found that the product molecules (proteins here) were formed much more rapidly than expected in the miniaturized system. In comparison with the DAPA system in particular, the reaction achieved was three to ten times faster, so that in the future, a protein copy can be concluded after about 15 minutes, instead of about 90 minutes as required in the present case.

With the DNA-to-DNA copy (the sample molecule is DNA and the product molecule is DNA), a DNA microarray could be created as a transfer storage of a previously unknown purity. The purity is so high that it presumably cannot even be detected by a sequencing process because that would be subject to more errors than the copy process that is used.

In a first subsection, this method thus allows a faster method of producing microarrays (in the form of transfer storages) that is less labor intensive, uses less material and yields a result of a greater purity that leads to a drastic cost saving while also allowing the creation of microarrays such as those that could not be produced with the previous methods or were feasible only by very time-consuming, expensive and labor-intensive methods that were not economical. Therefore, in the second subsection of the method, analyses that could not be carried out in the state of the art are possible.

In addition, it is advantageous that even DNA is sufficient to obtain a copy of a complete volume unit.

It is also preferable that the transfer storage is formed by a DNA-to-RNA copy. In one preferred embodiment, the DNA-to-RNA copy corresponds to an amplification copy. In this case, there is a DNA in the original that is amplified directly to RNA by means of an RNA polymerase. The resulting amplificates may then be bound directly to the copy. In another preferred embodiment, the DNA-to-RNA copy may be formed as a combination copy. In this case, the DNA of the original is first amplified as DNA by means of a DNA polymerase and then is amplified again by means of an RNA polymerase to form surface-bound RNA in a solid-phase reaction.

It is also preferable that the transfer storage is formed by a DNA-to-protein copy. In one preferred embodiment, the DNA-to-protein copy corresponds to a combination copy in which multiple reaction steps are connected in series. The DNA of the original is first transcribed to RNA by means of an RNA polymerase, and this RNA is then transcribed by means of ribosomes to a corresponding protein. The resulting protein then binds to the surface. In another advantageous embodiment, however, it is also possible to break the reaction down into two substeps. First, on the basis of the DNA of the original, RNA is produced by means of an RNA polymerase and is then bound to the copy surface. The copy may remain in this intermediate state until an enzyme mixture that uses the RNA as a template is added, then producing a corresponding protein therefrom, which is precipitated in the direct vicinity of the RNA.

It is also preferable that the transfer storage is formed by an RNA-to-protein copy. In one preferred embodiment, the RNA-to-protein copy corresponds to a combination copy because a corresponding protein is produced with this RNA by means of an enzyme mixture. The resulting protein is then transferred to the copy.

It is also preferable that the transfer storage is formed by an RNA-to-DNA copy. In one preferred embodiment, the RNA-to-DNA copy corresponds to a combination copy and a corresponding DNA is created from the RNA by means of reverse transcriptase. The DNA may then be transferred optionally directly to the copy or additionally amplified by means of a DNA polymerase and only then transferred. In such an embodiment, the RNA may advantageously be analyzed along with the resulting DNA.

It is also preferable that the transfer storage is formed by an RNA-to-RNA copy. In one preferred embodiment, the RNA-to-RNA copy corresponds to a combination copy because the RNA is derivatized by reverse transcriptase to form DNA. Then the DNA can be amplified again to RNA by means of an RNA polymerase or first amplified to DNA by means of a DNA polymerase and then this DNA is amplified to RNA by an RNA polymerase. The RNA is then transferred to the copy.

Analyses are then carried out. The analysis of the structure of the molecules may also cover their properties and may be carried out at different points in time:
The first storage may be analyzed before the copy.
The first storage, the transfer storage and/or the medium between the transfer storage and the first storage may be analyzed during the copy process.
The first storage, the transfer storage and/or the medium between the transfer storage and the first storage may be analyzed after the copy process.
A few individual ones or a plurality of species of molecules may be dissolved out of the first storage or the transfer storage and analyzed.
Substeps of the copy process such as amplification, derivatization or self-creation are analyzed.

The analysis depends greatly on the goal of the application. Conventional state-of-the-art analysis methods may be used. Established methods are the preferred methods and include fluorescence, luminescence, label-free detection, creation of stains that can be detected optically or redox-reactive species that can be detected electronically. Based on the spatial associability of the copies and the original, this association can also be achieved by all analyses among one another, so that the respective structure and properties can be associated with each molecule, its derivatives and amplificates, based on the analyses of the copies and of the original.

The following points can now be combined using the present invention in order to produce special arrays in a targeted manner. The transfer storages that are produced may then also be used for certain screening purposes.

In principle, each method according to the invention preferably requires the following four components:
Various sample molecules that are selected in a targeted manner are used as the source for creating the first storage, also referred to as the original,
Production of the originals may take place in various ways as described.
Then different copies are prepared therefrom in the form of the transfer storages
The analysis reactions then take place after or even during the previous method.

The method according to the present invention is preferably used for a random library or pool copy. This refers to any collection (a pool) of molecules, which belong either to the group of DNA or RNA or carry RNA or DNA and are of either artificial or natural origin or were created on the basis of a selection process or a mutation process. These molecules may also be derived from chemical libraries or display pools. A targeted selection of these sample molecules may be introduced and copied by any of the methods described here. A copy in the form of DNA, RNA or protein can optionally be produced therefrom. Then each of the copies may be used to optionally investigate a bond, an interaction, an enzymatic activity or a change in any of the aforementioned properties.

Furthermore, use for a display copy is preferred. On the basis of established display methods (yeast2hybrid, ribosome display, phage display, SELEX, etc.), first an enrichment is carried out with respect to a molecular target (biding partner, substrate, antibody, antigen, etc.). This step corresponds to the state of the art in the respective display method. However, after the first enrichment step, the pool that has been created can already be converted to an original according to the methods described here, and this original can then be copied many times in the form of DNA, RNA or protein, and thus the molecules that were enriched in the first step of the display are mapped as a microarray in their full number. Then a measurement may again be carried out on these copied microarrays with respect to the target. This then allows a much higher throughput of molecules investigated in comparison with the traditional displays and in particular it covers the entire pool. An enrichment may be carried out once again, if necessary.

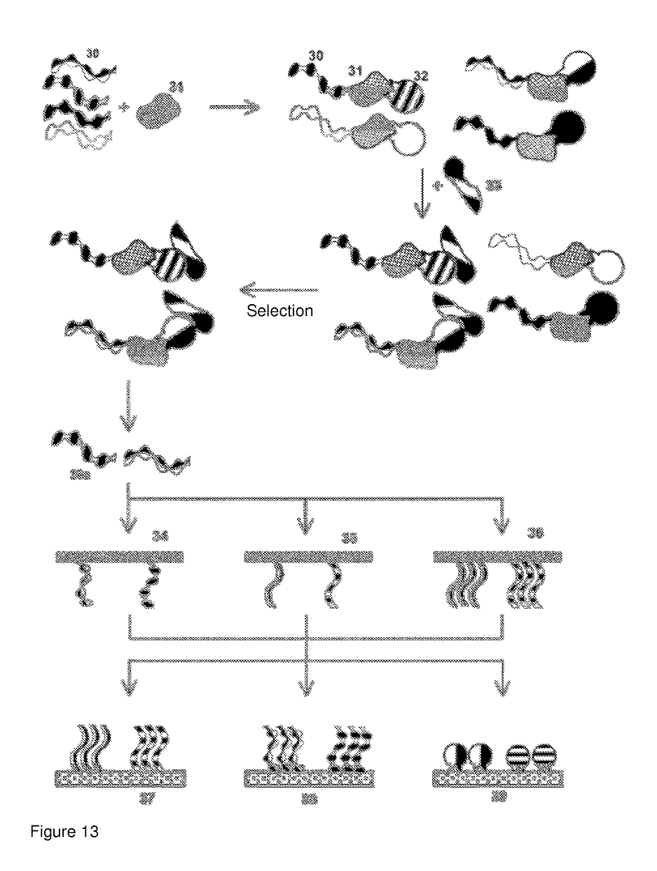

Furthermore, use in the ribosome copy is preferred. This use (cf. also FIG. 13) is derived from the ribosome display. To this end, as in the ribosome display, a bond to the desired target is created first and the binders are enriched. The enriched binders are then converted into an original according to one of the methods described here. The preferred embodiment here is the molecular storage, so that initially precisely one ribosome with the appended RNA strand or only the RNA strand or the DNA or cDNA strand derived therefrom is added in each position of the storage. Then an amplification is preferably carried out, so that the storage is preferably occupied with DNA. This ensures that the original will have long-term stability and that the degradation of the individual strands will not entail any loss of molecular information. Next, the original may optionally be copied to DNA, RNA or protein arrays. In one preferred embodiment, a protein copy is created and is then analyzed again with respect to binding to the target. The original or a DNA copy is sequenced, so that a DNA sequence can be associated clearly with any binding to the protein copy.

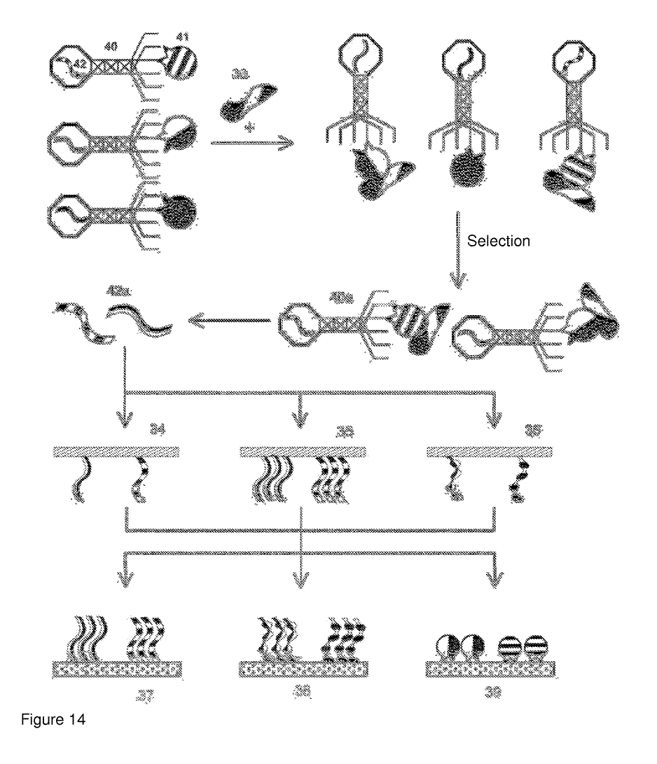

Use of the method in the phage copy is also preferred. This application (cf. also FIG. 14) is derived from the phase display. By analogy with the ribosome copy, the phage pool is enriched once with respect to the desired target and the phages are then transferred directly to an original. Next the steps are carried out as in the case of the ribosome copy. Then an amplification is preferably carried out, so that the storage is preferably occupied with DNA. This ensures that the original has long-term stability and the degradation of individual strands does not entail any loss of molecular information. Next, the original may optionally be copied to DNA, RNA or protein arrays. In one preferred embodiment, a protein copy is created and then is analyzed again with respect to binding to the target. The original or a DNA copy is sequenced, so that a DNA sequence can be clearly assigned to each bond to the protein copy.

Furthermore, use of this method in the antibody copy or the ScFv copy is preferred. In a special preferred embodiment of the display copy, the phages or ribosomes do not carry simple proteins but instead carry antibodies or parts of antibodies or artificial antibody-type constructs such as ScFv (single-chain antibodies). The method proceeds with these as done with the phage copy. The resulting protein arrays then carry antibodies, antibody parts and/or ScFv and are thus binders with respect to a target. This method may be used for optimization of antibody bonds.

Furthermore, a population copy is preferred. In this application a population of organisms (cells, viruses, bacteria) or macromolecules (vectors, plasmids, chromosomes, etc.) or molecular complexes (for example, interactions of a mixture of two ribosome displays in which, for example, one presents antibodies and the other presents antigens and thus carry two DNA tags per protein complex) is/are introduced into the original—also again preferably as a molecular storage. One or more molecules per storage are amplified in a targeted manner and stored in the form of DNA or RNA. Each line of the storage thus contains at least one molecule, which can be traced back to a source. Then copies may optionally be created in the form of DNA, RNA or protein and a sequencing of the original or a DNA copy may be carried out.

The following applications are also preferred:
The initial pool of sample molecules is analyzed for the number and type of mutations of one or more genes contained therein.
The initial pool of sample molecules contains B or T cells and the variations and combinations of the light and heavy chains of the B- and/or T-cell receptors are analyzed.
The initial pool of sample molecules contains polyploid organisms and the genetic variance of one or more gene sections is analyzed.
The initial pool of sample molecules contains interaction partners (each provided with a DNA tag) and the molecular structure of the binding partners can be inferred from the joint analysis of two DNA sequences (for example, the mixture of two ribosome displays of antibodies and antigen displays described above)
The initial pool of sample molecules contains one type of organism and the analysis provides information about the enzymatic or binding activity of one or more of the proteins contained therein.
The initial pool of sample molecules contains one type of organism and the analysis provides information about the enzymatic activity or binding properties of one or more RNA or DNA sections.

It is also preferable to use the method for the genome copy. Genomic DNA is obtained from one or more organisms and fragmented and then introduced into the original. This is also preferably carried out in the molecular storage. In the case of an upstream amplification, for example, emulsion PCR, which amplifies DNA to particles, a particle storage is used. The DNA in the original is then used to create DNA copies. The resulting array thus constitutes a genome array of the organism added. Such arrays could not previously be produced directly from the organism.

The transcription copy is also preferred. The RNA, preferably mRNA, is obtained from one or more organisms and then introduced into the original. This also preferably takes place in the molecular storage. a particle storage is used in the case of an upstream amplification, for example, an emulsion PCR, which first converts the RNA to cDNA and then amplifies it on particles. The RNA is preferably first stored in the cDNA in the original. This has a much higher stability than the RNA. Then copies are created in the form of RNA or cDNA. The resulting arrays are thus transcription arrays of the filled organism. Such arrays could not previously be produced directly from the original organism. In addition, the cDNA arrays thus created allow use in the field of expression analysis, whereas the RNA arrays created are used for binding analyses of promoters or transcription factors.

The proteome copy is also preferred. In this case the RNA, preferably mRNA or DNA is obtained from one or more organisms and then introduced into the original. The molecules stored there then consist preferably of DNA or cDNA derived from RNA. The preferred embodiment here is a molecular storage. A particle storage is used in the case of an upstream amplification, for example, an emulsion PCR, which first converts the RNA to cDNA or leaves the DNA as such and then amplifies it on particles. Next copies are created in the form of protein and then analyzed for activity in the form of binding, interaction or enzymatic reactivity. In addition, this array is the proteome of the organisms thus introduced as long as mRNA has been used. If DNA has been used directly, the copy maps more proteins than are present in the proteome. A complete proteome array could not previously be produced easily. ProtoArray 5.0 from Invitrogen, for example, covers only 9000 proteins from humans, who have more than 100,000 proteins.

The protein copy is also preferred. DNA which codes for the protein and/or mutations of the protein is obtained from a pool of sample molecules and introduced into the original. The molecules stored there are then preferably from DNA. The preferred embodiment here is the molecular storage. A particle storage is used in the case of an upstream amplification, for example, an emulsion PCR, which then amplifies the DNA on particles. Then copies are created in the form of protein and then analyzed for activity in the form of binding, interaction or enzymatic reactivity. Thus the array that is created is a general amino acid scan of the original protein. Such arrays could not previously be produced because 160,000 mutants would have to be produced for just one mutation scan of only four amino acid positions. This could not previously be implemented using the techniques available in the past.

In conjunction with optimization of molecules, the term scan is preferably understood to refer to the systematic variation of individual molecular building blocks. In an alanine scan, such as that used with proteins and peptides, one amino acid is replaced by alanine, which is mostly inactive, in a targeted manner, and the resulting product is tested. If a biochemically important amino acid was replaced, the biomolecule will exhibit a definitely reduced activity. Thus important and unimportant positions for the activity and/or properties of a molecule can be determined and/or estimated by means of the scan. For example, no information can be obtained about whether another amino acid instead of alanine would have a higher activity or an improved property in comparison with the original molecule.

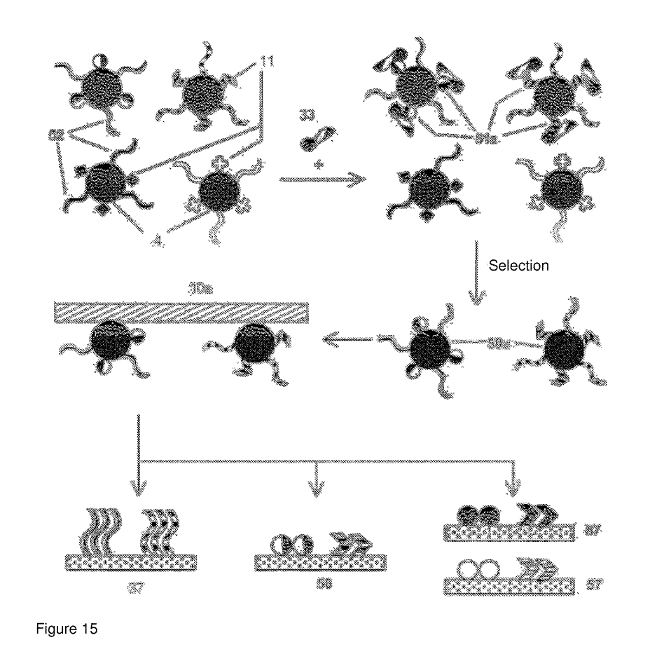

The preferred application in the field of combinatory chemistry copies is a very special combination (see also FIG. 15). Even during the synthesis of the chemical library, which preferably takes place on particles, in addition to the actual molecule, another “information molecule” is constructed in the form of DNA or RNA with each synthesis step. In doing so, the sequence of the DNA and/or RNA correlates clearly with the molecule thus constructed. After completion of this combinatory library, the particles are subjected to an enrichment with respect to a target. In other words, there remains a pool of particles that interact with the target. These selected particles are then preferably introduced into a particle storage, i.e., a particle transfer storage, as the original. Now the DNA or the molecules can optionally be transferred to the particle transfer storage. In this embodiment of the particle storage, no transfer is carried out at first and then multiple copies, which optionally carry the DNA and/or the molecules, are then created. The DNA may be amplified for this purpose. In the usual case, the molecules are released from the particle. One particle preferably carries definitely more molecules than are needed to create a copy, so that multiple copies of the molecules can be created. On the basis of the molecule microarrays, the binding to the target or structures similar to the target can be validated again, whereas the DNA copy is used to decode the sequence. Based on the correlation between sequence and structure, the molecular structure can be given for each spot on the molecule array. This is not possible with the combinatory split and mix libraries known in the past.

In addition, it is preferable that different species of sample molecules are bound to a particle.

It is preferable that the surface of the first storage and/or of the transfer storage is structured.

It is preferable that the sample molecules and/or product molecules are selected from the group including proteins, enzymes, aptamers, antibodies or parts thereof, receptors or parts thereof, ligands or parts thereof, nucleic acids, nucleic acid-type derivatives, transcription factors and/or parts thereof, molecules created with combinatory chemistry.

It is also preferable that the reaction step is carried out by means of DNA polymerase, RNA polymerase and/or a cell-free reaction mixture.

It is preferable that the structuring of the surfaces is selected from the group comprising cavities, elevations, cavities containing particles and/or elevations enclosing the particles.

It is preferable that the cavities are approximately the size of a biological cell. The cavities, especially preferably have a diameter of 5 to 250 μm, most especially preferably 10 to 50 μm. It has been found that this reaction step takes place particularly well in cavities of this order of magnitude. The yield is surprisingly better, the smaller the cavities.

It is also preferable that the first storage includes different physical, chemical and/or biochemical properties in different regions, preferably different volumes of the cavities, differences in pH, differences in salt content, temperature differences, different surfaces, differences in wettability, differences in electrical charge, differences in electrical, magnetic and/or dielectric properties, differences with respect to osmotic pressures, different additives, different biochemical ingredients.

It is preferable that during the reaction step, at least one species of sample molecule is released from the particles and/or the surface.

Furthermore, it is preferable that the analytical step e) comprises a label-free method, preferably RifS detection, iRlfS detection, Biacore detection, surface plasmon resonance detection, ellipsometry, mass spectrometry, detection of the increase in mass, detection of the change in the refractive index, detection of the change in the optical, magnetic, electrical and/or electromagnetic properties.

It is also preferable that analytical step e) comprises a method, which uses a label, preferably fluorescence measurement, detection by means of an absorbent and/or dispersing dye, mass spectroscopy by means of detection of an isotope label, detection by means of a molecule which changes the refractive index and/or the optical properties of the surface and/or of the solution.

It is also preferable that the analytical step e) comprises a method, which analyzes the solution above the surface of the first storage and/or of the transfer storage, preferably a turbidity measurement, fluorescence measurement, detection of an absorbent dye or stain and/or a luminescence measurement.

In another preferred embodiment, the invention relates to a change in a method of the aforementioned type, in a screening method for identification of transcription factors, transcription efficiency, transcription optimization, promoter efficiency, spliceosomes, restriction substrates, amplification system, codon optimization, protein functionality, enzyme functionality, enzyme optimization, isoenzymes, ribozymes, optimization of the reaction and/or optimization of the binding.

The preferred “total genome interaction screening” allows the identification of interactions at the level of the genome. A genome copy of an organism is created. Since all the DNA of this organism has now been mapped, any interaction partners or molecules may be added as sample molecules to this first storage. The following applications are preferred here:
DNA of a closely related organism or a mutant is added. Regions with bonds indicate identical DNA, whereas regions without bonds indicate clear differences between these species. These differences can then be used as markers for differentiation of the species.
RNA or cDNA from the same organism is added. The intensity of the individual spots indicates whether it is a gene and how strongly it is expressed.
cDNA is mixed in with a fluorescent dye 1 and a treated sample is mixed with a fluorescent dye 2 and then applied to the array. Based on the staining, it is possible to determine which gene was activated and how strongly.
Proteins are applied to the array. If there is an interaction or binding, then these are associated with the DNA sequence. Protein can therefore be identified as a DNA binder.

Use for transcription factor screening on a genome level is also preferred. This method allows identification of transcription factors at the level of the complete genome. A transcription factor is applied to a total genome array. By binding to individual spots, the sequence dependence of the transcription factor can be determined. Furthermore, additional parameters such as binding rate and binding strength can be determined by kinetic measurements, for example. Thus a genome-wide profile of its interactions can be obtained for each transcription factor.
If only one transcription factor is added, its profile can be determined. By varying the conditions such as temperature, pH or salt content, this dependence can also be determined throughout the genome.
If two transcription factors are mixed with different labels, for example, dyes or stains in the same ratio and then applied to the array, the interaction of the transcription factors can be deduced on the basis of the intensity of the individual spots and their staining. Sequences that are addressed by only one, none or both transcription factors can be identified. Variations of temperature, pH, salt content, etc. are also possible, so that a transcription factor can be intensified or diminished in a targeted fashion.
A mutation analysis can be carried out if a mutation is known for a transcription factor. Then the wild type and the mutation with different labels are also used on the array. Differences in binding behavior and thus gene activation can be analyzed on the basis of differences in color.

Furthermore, use for amplification screening on a genome level is also preferred. This method allows a deeper understanding of amplification systems for DNA and RNA. Individual molecules are molecule complexes of amplification systems (e.g., DNA amplification such as DNA polymerase, gyrase, helicase or RNA amplification) may also be applied to a total genome array. These molecules then preferentially bind to the positions which are necessary in amplification. These include, for example, the TATAA box for RNA polymerase or replication forks for the DNA polymerase as well as binding regions for helicases, gyrases, etc.
By adding the individual cofactors of the amplification system, it is possible to decode step by step where (in which sequence) and in the presence of which molecules the amplification complex is assembled.
By adding a cofactor with one stain and an iso-cofactor or a mutation with another stain, it is possible to ascertain on the basis of differences in color which genes are preferentially amplified. It is thus possible to draw conclusions about the expression of genes.

In addition, use for antibiotic screening on a genome level is also preferred. This is a special form of amplification screening which serves to identify new antibiotics and to characterize more specifically those that are already known. The antibiotics thereby identified serve as DNA or RNA amplification inhibitors and are therefore to be classified as bacteriostatics. To this end a human genome or a bacterial genome is created in the form of DNA. This DNA is preferably very long and/or is connected at its ends, so that “unrolling” of the DNA is very difficult to accomplish. A few DNA copies are created. Each DNA copy is then mixed with another antibiotic which suppresses or restricts the assembly of the DNA or RNA amplification complex or binding of a cofactor for the DNA or RNA polymerase.
To characterize known antibiotics such as the gyrase inhibitors nalidixic acid and ciprofloxacin with respect to topoisomerase II, the bacterial topoisomerase is labeled with one stain and the human counterpart is labeled with another stain. These proteins are mixed, combined with the antibiotic and applied to the array. Differences in color then reveal immediately where the bacterial amplification is restricted and where the human amplification is restricted. The interference in the amplification and/or the subsequent expression of the proteins can be inferred on the basis of these sequences. Thus various antibiotics can be coordinated with one another to inhibit the human system in the least invasive manner possible and to inhibit the bacterial system in the most effective way possible.
To investigate novel or unknown active ingredients, the human components of the amplification system are labeled with a stain and those of the bacterial system are labeled with another stain. These systems are then combined. First, a mixture is applied directly to a genome array. This represents the undisturbed system as a reference. Then an active ingredient is added to each mixture and testing is continued to ascertain when the human system is undisturbed and the bacterial system is disturbed. This case may involve a new antibiotic. In addition, however, each component of the amplification system may be tested individually with the new active ingredient. It is thus possible to identify the subunit that is attacked by the active ingredient, and it is then possible to show that this subunit no longer binds to the DNA.

Furthermore, use of the method for spliceosome screening on a genome level is also preferred. This method allows identification of splice variants. A total genome array is mapped as RNA. This RNA thus corresponds to the pre-mRNA. Then individual components or complete spliceosome complexes are applied to the RNA copy. This makes it possible to identify individual sequences that are recognized by the spliceosome. Furthermore, it is possible to assign its genome-wide interaction to each spliceosome. If a DNA copy is prepared of the RNA copy after treatment with the spliceosomes, additional sequencing is also possible. From knowledge of the original DNA and the DNA after treatment with the spliceosome, it is possible to obtain deeper knowledge about the sequence dependence and the splice variants. In addition, it is also possible through targeted mutations and/or addition of cofactors to prefer certain splice variants. This finding may be used in a targeted manner, for example, in culturing organisms or in differentiation of stem cells to implement or favor preferred splice variants.

Furthermore, use of the method for “total transcriptome interaction screening” is also preferred. This method allows identification of interactions at the level of the transcriptome. A transcriptome copy of an organism is then created. Copies are also created in the form of DNA, cDNA and even RNA. Since now all the cDNA as well as RNA of this organism has been mapped, any interaction partners or molecules can now be applied to this array. The following applications are preferred:
cDNA and DNA/RNA copy: DNA of a closely related organism or of a mutant is added. Regions with binding are indicative of identical DNA, whereas regions without binding indicate clear differences between these species. These differences may then be used as markers for the species differentiation. Furthermore, it is known thereby that these species have identical or related genes.
cDNA and DNA/RNA copy: RNA and/or cDNA of the same organism is/are added. The intensity of the individual spots indicates whether this is a gene and how strongly it is expressed.
cDNA and DNA/RNA copy: RNA and/or cDNA of another organism is added. Binding indicates related genes and therefore proteins, whereas spots without binding indicate that the other organism does not have the corresponding genes or that these are currently inactivated.
DNA/RNA copy: cDNA of a reference is mixed in with a fluorescent dye 1 and a treated sample is mixed in with a fluorescent dye 2 and then applied to the array. It is possible to ascertain on the basis of the staining which gene has been activated and how strongly.
DNA copy: proteins are applied to the array. If there is an interaction or a binding, then this may be assigned to the DNA sequence. It is therefore possible to identify protein as a DNA binder and potentially as an interaction partner in the form of signaling in that it interacts with this gene segment.
RNA copy: proteins are applied to the array. If there are interactions, they are thus RNA-interacting proteins. These proteins may be, for example, storages for RNA which are initially attached and then released as needed or assume a regulating function within the signaling.
RNA copy: siRNA is applied to the array. Interacting points indicate siRNA-based regulation mechanisms. It is possible to derive regulating mechanisms on the basis of these differences by adding individual siRNAs or a two color-labeled sample of siRNA from stimulated cells (color 1) and unstimulated cells (color 2).

Furthermore, use for the “total proteome interaction screening” is also preferred. This method allows identification of interactions at the level of the proteome. A proteome copy of an organism is created. If mRNA was used to create the original, the protein copy will reflect the proteome of the organism. If DNA was not used to create the original, then more proteins will be imaged than those that are present in the proteome. Since all proteins of this organism have now been mapped, any desired interaction partners or molecules may be applied to this array. The following applications are preferred here:
DNA of the organism or of a mutant is added. Regions with binding are proteins that interact with DNA. These may be, for example, histones, transcription factors or DNA-repairing proteins, etc.
DNA, RNA or the protein of the organism in one color is added and the DNA, RNA or protein of another organism or a mutant of this organism in another color is added. Commonalities and differences can be emphasized clearly on the basis of the color pattern. Relationships can be deduced. Differences allow the development of markers for clear identification of the respective species as well as the development of active ingredients, which can then be used in a species-specific manner.

In addition, the use of this method for active ingredient screening by addition of the active ingredient is preferred. This method allows identification of active ingredient on the basis of binding. Genome, transcriptome and proteome copies in the form of DNA, RNA and protein microarrays are created for these applications. A novel active ingredient or one that is already known is then added to these arrays. Spots to which this active ingredient binds are potential interaction partners of this active ingredient. Thus beyond a complete organism, the active profile of an active ingredient can be created. In combination with a measurement method which allows a kinetic measurement such as iRlfS or Biacore it is also possible to infer the binding strength.
If only one active ingredient is added, the active profile of that active ingredient can be determined.
If two active ingredients with different markers such as stain are added, cooperated effects can be inferred from the resulting coloration. Competition can be detected by control experiments with only one active ingredient in each case. This makes it possible to develop two active ingredients as a combination preparation, for example, and/or to ascertain whether two active ingredients address identical targets and binding pockets and therefore should not be administered in combination.
By adding two active ingredients of different labels, it is possible to identify adverse effects on the basis of interactions with unforeseen partners at the level of DNA, RNA or proteins and thus to combine them in such a way that the greatest possible effect is achieved with the least possible interaction with other partners and therefore adverse effects.

Use of the method according to the invention for promoter screening is most especially preferred. This application is unique. So far only statements about whether a promoter has a strong or weak effect have been possible in the state of the art. A true quantification is now possible for the first time through the present invention. This method allows an investigation of the effect of the promoter sequence on the quantity of protein produced. A DNA pool is produced. Each DNA contains a promote sequence and also codes for a protein. In the case of the promoter screening, there is a variability in the promoter sequence. This variability may correspond to the natural promoters of one or more organisms, either artificially or randomized. All DNA strands have identical sequences with respect to the coded protein, i.e., in the creation of a protein copy, the identical protein is formed by each sequence. Since the protein-coding sequence is identical, this means that the rate of production of protein depends only on the rate of initiation of the RNA polymerase and does not depend on the ribosomes. This may preferably take place as a molecular stored which is designed as a sequencing chip or as a classical DNA microarray. Then a protein copy is initiated and the amount of resulting proteins is analyzed directly in real time (for example, by iRlfS or Biacore). It is then possible to determine from this real-time data how quickly the individual promoters allow initiation of the RNA polymerase.
If only one RNA polymerase is used, a profile of the induction rate as a function of the promoter sequence can be determined.
If other polymerases may be used for additional copies, for example, then a sequence profile for these RNA polymerases can be created.
In addition, cofactors may be added and changes in the rates of production can be determined.

A sequencing chip is preferably a surface with which a sequencing is performed. Use of the FLX 454 chip from Roche is especially preferred because, due to its structure, it already has cavities that are advantageous for the copy technique.

Use of the method for transcription factor efficiency screening is also particularly preferred in particular. Such an application is impossible with the state-of-the-art methods. These methods are used for systematic investigation of the effects of transcription factors on the production of RNA and/or proteins. Just as in promoter screening, a DNA pool that does not differ in the region of the protein-coding DNA but is capable of binding in the region of the promoter and the region in which the transcription factors can bind. In other words, the identical protein is always formed. Since the protein-coding sequence is identical, this means that the rate of protein production depends only on the rate of initiation of the RNA polymerase and not on the ribosomes. First an original is created in the form of a DNA array. This may preferably be done as a molecular storage which is designed as a sequencing chip or as a classical DNA microarray. Then a protein copy is initiated and the amount of resulting proteins is analyzed directly in real time (e.g., by iRlfS or Biacore). It is then possible to determine from this real time data how quickly the individual promoters will allow initiation of the RNA polymerase.
First, identical data is obtained for the first copy as in promoter screening. Then, however, additional copies are created, which then alter the enzyme system through the addition of transcription factors (inhibiting as well as activating). Due to the changes in the rates of creation of the proteins, there may be a correlation between the promoter sequence and the strength of the transcription factor. It may be assumed that sequence dependencies are very clearly recognizable in this structure and are to be quantified precisely. This measurement has not previously been possible in any other system.
Transcription factors of other species may also be used and may thus analyze the interspecies compatibility of the individual biochemical mechanisms in vitro. This is of interest in particular for the production of cell-free mixed systems which can in turn be used for cell-free production of proteins from DNA (for production of protein copy, among other things).

Furthermore, it is preferable to use the method according to the invention for codon optimization. This has been possible only with an extremely great effort in the prior art. The use according to the invention is for optimization of a DNA sequence for improved biosynthesis. As in promoter screening and transcription factor efficiency screening, a DNA pool is constructed in which each DNA strand carries an identical promoter sequence. The differences between the DNA strands consist of the protein-coding sequence. Although they code for the same amino acid sequence, they differ in the codons. In other words, the same protein is always produced but different tRNA pools are used to produce it. For the initiation of RNA polymerase, the same promoter is always used initially in an identical manner and at the same rapid rate for all DNA sequences. However, the use of different tRNA pools means a difference in synthesis rate. The difference in the rate of production thus depends only on the codon sequence. First an original is created in the form of a DNA array. This may preferably be done as a molecular storage which is designed as a sequencing chip or as a classical microarray. Then a protein copy is initiated and the amount of the resulting proteins is analyzed directly in real time (for example, by iRlfS or Biacore). It is then possible to determine from such real-time data which choice of codon is optimal for high-speed synthesis. These results are of interest in optimizing “codon usage” in the production of recombinant proteins in cells in particular.

Furthermore, it is preferable to use the method according to the present invention for global antibiotic screening by direct inhibition. This method also serves to identify antibiotics. Instead of investigating the assembly of the human and bacterial amplification complexes in the presence of the antibiotics, as is the case with the antibiotic screening on a genome level described here, an original that contains human and bacterial DNA is used (including binding sites for transcription factors and promoter sequences). Identical DNA copies are created first from this DNA original. Then cell-free expression systems (human and bacterial) are each mixed with one active ingredient and a protein copy of the DNA copy is produced. This is analyzed quantitatively to determine how much of which protein is formed. If one of the active ingredients interferes with the production of protein or the upstream amplification of RNA in any way, this is revealed by the reduction in or failure of the corresponding protein spot to appear. Active ingredients that inhibit or suppress the production of protein in a manner which depends on the sequence and/or on the expression system make themselves known due to the omission or weakening of individual spots and/or the omission or weakening of the complete protein copies. Active ingredients that inhibit the bacterial system and do not influence the human system are thus potential antibiotics that inhibit protein production in bacteria in a direct manner. The active ingredients thereby identified can then be subjected to an active ingredient screening in detail.

Use of this method for global antibiotic screening by substrate inhibition is also preferred. This method is also used for identification of antibiotics. Instead of analyzing the assembly of the human and bacterial amplification complexes in the presence of the antibiotics, as is the case in antibiotic screening on a genome level, an original that contains human and bacterial DNA is used (including binding sites for transcription factors and promoter sequences). Then optionally DNA, RNA and protein copies are created. Substituting molecules for the original monomers are added to the respective enzyme mixes for the creation of the respective copy. These substituents are then potentially incorporated into the DNA, RNA or proteins instead of the original monomers. Of the copies produced at the level of RNA and/or DNA, protein copies are then produced again. If one of the substituents used has an inhibiting effect, this will be apparent due to the fact that little or no protein is created, i.e., less than that in comparison with the unsubstituted enzyme mixtures. If less protein can be formed in individual positions, it is possible to deduce that there is a sequence-specific inhibition. If little or no protein is formed in general, then a systematic inhibition of protein synthesis must be assumed. The substituents identified in this way can then be analyzed again in vitro and in vivo to determine their effects. If the inhibition occurs to a greater extent in the bacterial system, then this is a potential antibiotic.

In addition, the use of the method for substituent screening is also especially preferred. This application is also unique in comparison with the prior art because each substituent had to be synthesized individually in the past. No copying process has yet been described in this context. This method makes it possible to discover activators, inhibitors and equivalent substitutes. To this end, the corresponding molecular domains are coded for a receptor or binding partner at the level of the DNA, and these DNA sequences in the form of a DNA array are used as the first storage, preferably as a molecular storage. Then copies are made of this original in the form of DNA, RNA or protein. With the corresponding enzyme systems, monomers are substituted, preferably completely. As a result, instead of the original monomer, only the substitution is incorporated. Several copies each with different substituents are created. Then the receptor is added to the individual copies and both the binding and its activation are measured. Spots that no longer have any binding do not have any effect. Spots with binding but without any change in activity contain a molecule that can be used as a substitute. Spots with increased or reduced activity contain molecules that can be used as activators and/or inhibitors. The following applications of substitution are preferred here:
By incorporation of unnatural DNA, RNA or amino acid building blocks, the activity of the molecule can be improved/reduced or a substitute for the previous molecule can be found.
By incorporating of substituents, it is possible to alter molecular properties. This refers in particular to solubility, toxicity, pH and thermal stability, charge, stability with respect to proteases, DNases or RNases.
When the active ingredient thereby discovered has the same activity but a delayed degradation or metabolism, it may be administered in a lower dose.
Adverse effects can be reduced by also testing for additional interactions but are responsible for adverse effects and then selecting the substituents, so that these interactions are minimized at the same level of activity.

Furthermore, use in a growth factor substituent screening is also preferred. This method is a special case of substituent screening. Growth factors, in particular EGF and VEFG [sic; VEGF] are usually highly activating in the case of tumor cells. It is therefore of particular interest to discover molecules that bind to the EFG [sic; EGF] and/or the VEGF receptor. In this particular case, the receptor may even be activated initially because another enzyme system deactivates a receptor that has been active for a longer period of time. If there is no renewed activation due to the binder remaining in or on the receptor, then it will remain permanently deactivated. This means that the growth of the tumor is slowed down and the prospects for a cure are improved significantly when combined with a therapy.

Known and presumed interaction partners of the EGF, VEGF and other growth factor receptors are therefore coded on a DNA level. These interaction partners are mainly proteins. Therefore, a DNA array is first produced as an original, preferably in the form of a molecular storage. Then enzyme mixtures are prepared to create a protein copy, in which at least one amino acid is depleted and replaced by an artificial different amino acid or in which a codon is replaced by another artificial amino acid. This means that the protein copy then has the artificial amino acid everywhere instead of the original amino acid. These molecules have the same or a similar 3D structure in principle and are thus potential interaction partners of the receptors. Then the receptor is added to the protein copy and checked for whether binding occurs. This may take place directly by a real time measurement such as iRlfS or Biacore. After binding the activity of the bond receptor is measured. In the case of the EGF and/or VEGF receptor, this may be detected by a phosphorylation reaction. To this end, radioactive ATP is added to the copy to which the receptor is already bound. Active receptors convert this ATP and bind the radioactivity to themselves. It is possible by means of autoradiography to quantify how much ATP has been bound (applying a photographic film, then developing and analyzing the opacity of the film) and how active the receptor is. It is thus possible to evaluate whether binding has occurred and how this has affected the activity of the receptor. The respective protein sequence can be determined on the basis of the DNA original and then the respective artificial amino acid can be determined on the basis of the additive to the protein copy. It is thus known which substituent has an activating or inhibiting effect and can potentially be used as a tumor medication or as a growth agent.

Use for enzyme screening has also exhibited special advantages and is therefore is therefore preferred. This application makes it possible to select from a variety of enzymes those which have the most advantageous properties. To do so, all the enzymes in question are coded at the level of the DNA, and a DNA array is created as an original, preferably as a molecular storage. This corresponds to a pool copy as described above. However, cell cultures or microorganisms may also be cultured in a targeted manner with a substrate which must be converted in order to survive. To this end, the original enzyme is usually known and a combined mutation-selection pressure is applied to the organism. The DNA sequence of the enzyme in question is thus altered by mutations. However, these mutations are only minor in most cases and the DNA remains accessible to targeted PCR, so that a DNA original can be created according to the population copy of a population of organisms which have mutations in the protein/enzyme in question. By means of a protein copy, the enzymes are then created as an array. By adding the desired substrate of the enzyme and using a detection reaction which correlates with the enzyme activity and/or the conversion of the substrate, it is possible to detect the activity of each enzyme. The following applications are especially preferred here:
Different substrates are added to the individual copies and the substance conversion of the substrate is detected in the respective spot. In this way a profile of the substrate specificity and the activity is obtained for each of the enzymes created.
The same enzyme is used everywhere within a property storage. Because of the different properties in the different positions, for example, different pH, salt content or temperature, it is possible to detect under which conditions the enzyme functions optimally.

Use for enzyme optimization is also preferred. If an enzyme is already known, it can now be optimized. To do so, the coding DNA of the enzyme is modified systematically or randomly at individual positions, so that individual ones or multiple amino acids are replaced. The DNA pool thereby created is then created as a DNA original according to the pool copy, and then corresponding protein microarrays, which then contain all the desired mutations of the enzyme, are created by means of the protein copy. Next the copy is incubated with the substrate of the enzyme. Enzyme variants with a high activity then convert this substrate, thereby generating a signal more rapidly than do the less active enzymes. Then the most active enzyme can be selected on the basis of the sequencing of the original or a DNA copy. In addition, it is possible to test various copies under different conditions, so that here again, a profile of each enzyme can be prepared.

Furthermore, it is preferable to use the method according to the present invention for stability screening. This method serves to improve the “stability” of molecules and/or provide a higher stability with respect to external influences as well as decomposing ambient conditions and also enzymatic activity. Four strategies can be pursued here:
The original molecule is mutated systematically or randomly at individual positions or in multiple positions, so that one monomer is replaced.
Individual ones of the natural monomers are replaced by artificial monomers in a targeted manner.
A chemical component of the monomers is altered, so that the entire molecule is stabilized.
The original molecule is shortened and/or lengthened with flanking sequences to thereby achieve a greater stability.

Regardless of whether the molecule is DNA, RNA or protein, our three strategies can be used separately or jointly. If the stability can be detected by a bond, then a pool of up to 10¹⁵ or more different molecules may be used to enrich the molecules that form bonds. It may be assumed that more stable molecules can maintain their bonding ability for a longer period of time. The remaining pool should contain enough molecules, so that a pool copy can be created. This is first implemented at the level of DNA. Depending on the desired molecule, then DNA, RNA or protein copies are created. These copies may then be exposed to various decomposing influences such as high/low pH, aggressive chemicals, high temperatures, enzymatic activities, etc. Each spot on the copied microarray is then analyzed and its decomposition is measured in real time. Stable molecules are characterized by a much slower decomposition.

Use for DNase/RNase stabilization is also preferred. This method serves to increase the stability of DNA and RNA. The following strategies may be used for this purpose:
The original molecule is lengthened with flanking sequences to thereby achieve a higher stability. Secondary and tertiary structures which can no longer be attacked by RNases and DNases are formed by the flanking sequences.
Individual ones of the natural monomers are replaced by artificial monomers in a targeted manner. One of the four building blocks here can be replaced by an artificial DNA or RNA base.
A chemical component of the monomers is altered, so that it stabilizes the entire molecule. In the case of RNA and DNA, so-called PNAs are very well known. In this case, the phosphate may be replaced by an amino acid. This replacement protects against decomposition by RNases and DNases but still allows full functionality. However, other modifications of the sugars, the phosphates or the bases are also applicable.

To do so, the original DNA sequence and/or RNA sequence is provided with flanking sequences. These are generated systematically or in a random process. The resulting variants are created as a pool copy in the form of a DNA array as the original. Then multiple copies are created in the form of DNA or RNA microarrays. Other artificial monomers may be added already at the time of creation of the copies or chemical modification may be performed after creation of the copy. Optionally a label which detects whether the complete DNA or RNA strand is intact may also be introduced already during their creation (e.g., fluorophores or a fluorophore-quencher pair). The copies thereby created are then exposed to the decomposing influence. In the case of RNases and DNases, they are applied directly to the microarrays. DNA and/or RNA strands of a low stability decompose, which is detectable in the case of incorporated fluorophores by a reduction in fluorescence (by an increase in fluorescence in the case of a fluorophore-quencher pair). Within an array, it is thus possible to determine the stability of individual flanking sequences in a sequence-specific manner. If spots having the same sequence on different arrays are compared with one another, the stabilizing effect of the individual monomers or chemical modification may permit a conclusion to be drawn. The most stable possible DNA and/or RNA strand can be derived from the combination of sequence dependence, substitution of individual monomers and chemical modification.

Protein stabilization is also a preferred field of application. This method serves to increase the stability of proteins. The following strategies may preferably be employed to do so:
The original protein is lengthened with flanking sequences to thereby achieve a higher stability. Secondary and tertiary structures formed by the flanking sequences can no longer be attacked by proteases or they cover regions of the protein that would otherwise be subject to attack.
Individual ones of the natural amino acids are replaced by artificial amino acids in a targeted manner and these artificial amino acids can then no longer be degraded by proteases.
A chemical component of the amino acids is altered, so that it stabilizes the entire molecule. For example, a chemical modification of the peptide bond is conceivable here.

To do so, the original protein sequence is prepared in the form of coding DNA and is provided with flanking sequences as needed and/or individual ones or several amino acids are replaced. These variations are generated systematically or in a random process. The resulting variants are created as a pool copy in the form of a DNA array as the original. Then multiple copies are created in the form of protein microarrays. Artificial amino acids may already be added in the creation of the copies or chemical modifications may be performed after creating the copy. Optionally a label may introduced already during the creation of the copy, said label being capable of detecting whether the protein is intact (e.g., fluorophores or a fluorophore-quencher pair) within an array. It is thus possible to determine the stability of the individual flanking sequences or amino acid exchange positions in a sequence-specific manner. If spots with the same sequence in different arrays are compared with one another, conclusions can be drawn about the stabilizing effect of the individual monomers or chemical modifications. The most stable possible protein can be derived from the combination of sequence dependence, substitution of individual amino acid and chemical modification.

Furthermore, use for antibody stabilization is also preferred. This method allows stabilization of antibodies. Antibodies are increasingly used in the therapeutic and diagnostic fields. Long-term stability in storage is advantageous for this application. The original DNA sequence, which codes for the antibody in the positions that are not relevant for the binding capability of the antibody to the antigen, is therefore varied in those positions. These variations may be inserted in a targeted or random manner. The resulting DNA pool is then created as the DNA original. Protein copies thereof are then prepared. These protein microarrays form a mutation library of the original antibody. One of the copies is used for binding analysis to detect whether the mutations used have no effect on the binding ability of the antibody. Then the copies are incorporated and a set of copies is then tested for binding at regular intervals. It is possible in this way to determine which variant of the antibody still has a high activity even after a long storage time. In parallel with this, the antibody array may also be exposed to various proteases to ascertain how stable each variant is to decomposition by proteases. An optimized antibody sequence having long-term stability can be derived from these results.

Use of this method for substrate screening is also particularly preferred. It is possible by means of this application to ascertain, for a given enzyme, which type of substrate it can convert at the level of DNA, RNA or protein. To do so, all the known substrates are first coded at the level of DNA, and mutations are then introduced into these substrates. The resulting DNA pool is prepared as a DNA original. Then, depending on the required substrate, copies are prepared in the form of DNA, RNA or protein arrays. Generation of signals can already be integrated into the production here. For example, fluorophores may be incorporated randomly or in terminal position or a fluorophore-quencher pair may be created. This may then be implemented by whole-area application of a fluorophore to the surface, for example. The molecules thereby created then carry the quencher in terminal position at the greatest possible distance from the surface. Then the enzyme can be added to the copy thereby created. Depending on the class of the enzyme, a corresponding signal may be generated, which detects the enzyme activity by the fact that the respective spot on the microarray has been altered. In splitting the substrate, for example, a fluorophore or a quencher may be split off. In a redox reaction a dye may be altered or in a ligation a fluorophore or a quencher may be inserted and the fluorescence thereby altered. Spots that change are thus a substrate of the respective enzyme. Since the sequence can be ascertained on the basis of the original or a DNA copy, it is possible to deduce the respective DNA, RNA or protein sequence. In this way it is possible to determine the band width of the substrate for an enzyme.

Use of the method for protease screening is also preferred. Using this method, the corresponding proteins that can be cut off from them can be found for different proteases. The sequence dependence of flanking sequences can be ascertained in particular. A DNA pool is therefore optionally created on substrate-coding DNA, or a total genome, a complete transcriptome or a complete proteome array may be used for this purpose. In any case there is initially an original on a DNA level. Then corresponding copies are made of this original in the form of protein by means of protein copying. The copy is then incubated with the protease. If a spot contains a protein that is decomposed by the protease, a signal is generated there. This may take place, for example, through an increase in fluorescence when a quencher is split off or through a decrease in fluorescence when incorporated or terminal fluorophores are split off because the protein change has been degraded. On the basis of the comparisons of the DNA sequence and the protein sequence derived therefrom it is then possible to determine which proteins are decomposed by the protease. If detection of the kinetics is possible, information can also be obtained about how strongly flanking sequences or replacement of one amino acid by another can have influence the catalytic activity of the protease. If a total proteome copy has been used, a conclusion can be drawn about which proteins of the proteome are decomposed by this protease.

Furthermore, the use of this method for kinase substrate screening is also preferred. Using this method it is possible to prepare a substrate profile for kinases. The sequence dependence in particular can be determined. To do so, a DNA pool is optionally created on protein-coding DNA, or a total genome, a total transcriptome or a total proteome array may be used for this purpose. In each case, there is first an original on a DNA level. Then by means of protein copy, corresponding copies are made of this original in the form of protein. The copy is then mixed with kinase, and in one preferred embodiment, combined with radioactively labeled ATP. In other preferred embodiments, other methods of generating signals are also conceivable (for example, by fluorescence labeling or by electrical detection of the change in pH during phosphorylation). If a spot serves as a substrate of the kinase used, then the radioactive phosphate is bound directly to the protein. Then the radioactivity in each spot can be quantified by means of autoradiography. Particularly well accepted substrates of kinase have a particularly high radioactivity. Thus all the substrates of this kinase can be detected for the pool of selected proteins or throughout the proteome. If artificial amino acids have also been added in creating the protein copies, information can also be obtained here about the acceptance of kinase for such substrates. It is thus possible to make an assignment of protein sequence and kinase activity for this kinase.

Use for phosphatase-substrate screening is also particularly preferred. Using this method it is possible to prepare a substrate profile for phosphatases. The sequence dependence in particular can be determined. This method represents an inversion of kinase substrate screening in principle. A DNA pool of protein-coding DNA is therefore optionally created, or a total genome, a total transcriptome or a total proteome array may be used for this purpose. In any case, an original is obtained first on a DNA level. Then by means of protein copy, corresponding copies are created thereof in the form of protein. In one preferred embodiment, the copy is labeled with radioactive phosphate. In other embodiments, other forms of generating signals are also conceivable (for example, by fluorescence labeling or by electrical detection of the change in pH during a dephosphorylation). Thus each spot has an initially high radioactivity. If one spot serves as a substrate of the phosphatase used, then the radioactive phosphate is split off from the protein. The radioactivity in each spot can be quantified by means of autoradiography. Especially well accepted substrates of phosphatase have a particularly low radioactivity. Thus all substrates of this phosphatase can be detected for the pool of selected proteins or throughout the proteome. If artificial amino acids have been used in addition in creation of the protein copies, information can be obtained here again about the acceptance of the phosphatase for such substrates. It is thus possible to make an assignment of protein sequence and phosphatase activity for this phosphatase.

Restriction substrate screening also constitutes a preferred application. Using this method it is possible to conduct testing throughout a genome to determine where a restriction enzyme manifests corresponding decomposing activity. To do so, a total genome copy is created as a DNA original. This original is then copied in the form of DNA microarrays. Thus all the DNA sequences of the genome are present. Spots that are decomposed by the restriction enzyme can be detected based on the increase in fluorescence (when splitting off a terminal quencher) or by the decrease in fluorescence (by splitting off a terminal fluorophore). It was thus possible to detect which sequences are decomposed by the restriction enzyme throughout the entire genome.

Furthermore, use for isoenzyme differentiation is also preferred. If there are several isoenzymes of one enzyme, there may be a differentiation of the substrate dependence in that identical microarray copies are incubated with one isoenzyme each and a substrate profile is created by means of the methods described here for substrate screening, protease screening, kinase substrate screening, phosphatase substrate screening and/or restriction substrate screening. A comparison of these profiles with one another thus makes it possible to discover in a targeted manner those spots that are converted especially well by one of the isoenzymes and those that are converted especially poorly by another isoenzyme. This DNA sequence is then modified again in a targeted manner or randomly in individual positions. The resulting DNA pool is then in turn stored as a DNA original and corresponding microarray copies are prepared. These are each then exposed again individually to the isoenzymes. Based on the renewed mutations of the substrate which has already supplied the best differentiation between isoenzymes, there is now a good chance purely statistically of finding an even better substrate, which will be accepted well by one of the isoenzymes and poorly by the other. In this way it is possible to generate a substrate specifically for only one isoenzyme.

Through the incorporation of artificial monomers during the creation of the copy, it is also possible to generate substrates that are almost impossible to convert and are accepted by only one of the isoenzymes. With a high affinity accordingly, this unconvertible substrate forms an inhibitor for this enzyme. This discovery of isoenzyme inhibitors is of great interest in particular in the development of drugs.

Furthermore, the ribozyme copy method may also be used. This method makes it possible to detect catalytic activities of RNA. Ribozymes have catalytic activity and have properties like enzymes but consist of RNA. First a DNA pool that codes for potential ribozymes is created. This DNA pool is then converted into a DNA original and then RNA copies are prepared. Next one substrate is added to each of these arrays. Spots that do not show any catalytic activity with respect to the substrate generate a signal based on the conversion of the substrate. This may be, for example, splitting off a chemical group, which changes the color of the molecule. Based on the sequencing of the DNA original or a DNA copy, the sequence of the RNA can be derived and from that it is also possible to determine which sequence has which catalytic activity.

Use of the method according to the invention for display screening is most especially preferred. One particular advantage is that this screening may be used as the so-called “end stage” for all conventional displays. This method constitutes an expansion of the throughput of analyzed molecules for the respective display method, selection method or enrichment method for DNA, RNA or proteins. It is possible to create an enriched pool of 10⁶ or fewer molecules having the desired properties can be created by means of the respective method, which usually starts with a pool of molecules containing 10⁹ or more different molecules. This pool of molecules having enriched properties is reshaped to yield a DNA pool (RNA by means of reverse transcriptase to DNA, proteins of a display always belong with the generating DNA, this can be arranged by means of PCR to form a DNA pool). This DNA pool is then created as a pool copy according to and/or as a display copy and/or its subvariants as a DNA original. Then corresponding copies in the form of the molecules required are created of this DNA original. These copies may be DNA, RNA or protein. Each copy may then be investigated with respect to another molecular property. For each individual of the DNA pool and/or of the originally enriched pool, the respective set of properties can be assigned from this assignment of each point of each copy to the original DNA sequence. Then the molecule that has the best combination of desired properties can be determined from this set. Since many different properties can be detected by means of the copies and since one copy carries many individual molecules, a definitely higher conversion of investigated molecules is generated by using this method than has been possible with the previous methods.

It is also preferable to use the method according to the invention for antibody screening from artificial sources. This method facilitates the display screening of antibody libraries, in particular the phage display with antibodies. First, an enrichment of the phages with respect to the antigen is performed using a phage display library with artificial randomized antibodies or antibody fragments, so that of the initially often up to 10¹⁵ different antibodies, only less than 10⁶ different antibodies and the respective DNA strains remain. A DNA original is then created from this enriched DNA pool. The DNA original is then mapped in the form of DNA, RNA and/or protein copies. By means of transfection methods, it is then possible to transfect the DNA or the RNA into cells and thus excite them to production of antibodies. In parallel with that, the protein copy which contains the antibodies can be investigated for binding to the antigen. Spots having a particularly strong signal have a particularly strong binding to the antigen. Based on the sequencing of the DNA original or a DNA copy, it is possible to deduce the amino acid sequence of the antibody. If DNA or RNA has been recovered, it may be used directly for transfection. It is therefore possible to then recreate the antibodies that have been identified bond well directly in cells. This method makes it possible to achieve an improvement in the throughput of the analyzed molecules as in all display methods. This case involves antibodies. Advantageously only a single enrichment with respect to the antigen must be performed instead of the three to four rounds of a phage display that would otherwise be customary. Furthermore, at the end of the analysis, one has data on up to 10⁶ antibodies instead of the usual 10² to 10³. This embodiment thus constitutes an improvement in the previous phage display with antibodies.

It is also preferable to use the method for antibody sampling from organic samples. With this method, antibodies to antigens can be identified from organisms in a targeted manner. For example, B cells are obtained from an organism that was exposed to an antigen. Each B cell then has a different antibody coded in itself. To this end, it is necessary to connect the variable sequence part of the mRNA of the light chain and the heavy chain of each cell to one another. The B-cell population can be created directed as a population copy from the DNA original. The DNA is then the cDNA derived from the mRNA of the antibody. However, it is also conceivable for the cells to first be isolated physically and processed, for example, in a droplet emulsion and for the mRNA to be transcribed to cDNA in each of these compartments and then for the cDNA of the light chain and the heavy chain to be linked together to form a DNA strand. The DNA pool created in this way can then be converted to a DNA original. In the preferred embodiments, either the light chain and the heavy chain are present in the full length (design 1) or the construct thus created corresponds to an ScFv (design 2), in which the variable regions of the light chain and the heavy chain are connected to one another by means of a short spacer. In both cases, the DNA original or a DNA copy is sequenced and protein copies are prepared. The resulting protein arrays contain the antibodies (in design 1) or the ScFv antibodies (design 2). These antibody arrays are then incubated with the antigen to which the organism had been exposed. Antibodies to this antigen then have one bond to the antigen. In this way, the respective sequence of the binding antibodies can be identified for this antigen and an ScFv library can also be obtained.
In addition, the antibody arrays thus created can be incubated with different antigens and tested to determine whether any additional binding activities are present.
Likewise, a lysate from the infective antigen or antibody may be brought to the antibody arrays and thus all the antibodies to the antigen may be determined.

This method is particularly advantageous because it makes it possible to obtain a plurality of antibodies systematically, to characterize them and to elucidate the DNA sequence for subsequent use.

Furthermore, the use for antibody optimization is also preferred. If an antibody to an antigen is known, this antibody can be further optimized with regard to its properties and binding capabilities. To do so, a DNA pool containing mutations or substitutes in certain positions or in random positions is created based on the coding DNA. This DNA pool can then be enriched according to a display method with respect to the desired properties of the antibody (greater solubility, better stability at a modified pH or salt content, etc.) and is then processed to yield a DNA original, which is then mapped again by means of the protein copy to yield antibody arrays. The antibody arrays thereby created are then incubated with the antigen under the desired conditions (concentrated solution, altered pH or salt content, etc.) and detected. The spot with the strongest bond constitutes the antibody with the best desired property. Based on the sequencing, the DNA sequence and thus the amino acid sequence of the antibody can be decoded. Using this method, it is then possible to compare a large number of antibodies directly with one another and thus select the best from a large range of possibilities. Previous display methods are greatly limited in particular in the number of antibodies characterized so it often occurs that the best antibody is not detected. Due to a higher throughput, the chance of detecting the best antibody is greatly improved. This method allows optimization of antibodies, antibody constituents or artificial antibodies.

Use of the method for antibody optimization of ScFv is also preferred. Using this method, a pre-existing ScFv (single chain antibody) is optimized with respect to its connecting chain. In the case of ScFv, there are only the two variable binding regions of an antibody and they are linked together by a very short connecting chain. Without this connecting chain, the affinity of the short variable chains is too low, so the complex simply disintegrates. The connecting chain thus serves the purpose of stabilization. To this extent, it is of great interest to optimize this connecting chain in such a way as to achieve the greatest possible affinity for the antigen. Therefore, a DNA pool which codes for the ScFv is created. The DNA is always identical in the range of the variable regions, and mutations are inserted randomly or in a targeted manner in individual positions or in several positions only in the region of the connecting chain. The DNA pool may optionally be enriched by means of a display method, so that ScFv having the highest possible affinity are encoded or are used directly. A DNA original is created and protein copies are generated from it. Then all of the ScFv arrays thereby obtained contain mutations. Then the antigen is added to these arrays and the binding is measured. Spots having a particularly high binding also have a particularly high affinity for the antigen. Based on the sequencing of the DNA original or a DNA copy, the amino acid sequence of the connecting chain having the highest affinity can be determined. Likewise, a ranking list of affinities can be created and these assigned to the sequences. A system for assigning an affinity to the respective sequence can be derived from similarity comparisons. This system may be used for predicting binding affinities of ScFvs with respect to other antigens. On the whole, this method allows optimization of the connecting chain in that a plurality of variants is investigated and a system is derived.

It is also particularly preferred for the method according to the invention to be used for epitope screening for development of a vaccine. The method can be preferably be used to produce all the peptide vaccines. It is possible in this way for the first time to perform vaccine production within a few days. Using this method epitopes can be derived at the level of the DNA, RNA or proteins which serve as immunogens and are thus suitable for use as vaccines. This requires an organism that has an immune system. This organism is exposed to a parasite, a bacterium or a virus (immunogen). In the case when the organism survives, there are corresponding bodies in its blood stream which were generated by means of an immunologic defense and then make the organism immune to the immunogen. Next a tissue sample and a blood sample are taken of this organism. The antibodies and the B cells from the blood sample are then purified. The tissue sample is transferred to a cell culture and again infected with the immunogen. This infected tissue sample is then harvested and a total genome, a total transcriptome and a total proteome are produced from the tissue sample. The arrays thus also contain the molecules formed by infection in addition to containing the usual molecules for that organism. The purified antibodies are then added to these infection arrays. If immunogens are present at the level of DNA, RNA or protein, then the antibodies will bind to them. Thus each spot to which the antibodies bind becomes a potential epitope of an immunogen. By sequencing the DNA original, the DNA, RNA or protein sequence of the immunogen can be elucidated. Furthermore, it is possible to release the infected immunogens or their DNA from the DNA original or one of the copies in a targeted manner. The immunogens therefrom can then be purified or created. These immunogens may then be added to the resulting B cells. B cells that bind the immunogen are activated and begin to divide. It is thus possible to create a cell culture which produced specifically the antibodies that defend against the immunogen. Thus for development of vaccines, both the immunogen itself is available for active immunization as well as passive antibodies being available for passive immunization. This combination is unique and allows the development of vaccines within one week.

Epitope screening is preferably also used to determine the autoimmune status. Using this method, it is possible to clarify whether there are epitopes that trigger an autoimmune response at the level of the DNA, RNA or proteins. This method corresponds largely to the epitope screening for vaccines. The organism to be investigated is itself the organism that suffers from an autoimmune reaction. In any case, the organism has in its blood stream the corresponding antibodies, which have been generated by means of an immune response and are inducing the organism to develop an autoimmunity. Then a tissue sample and a blood sample are obtained from this organism. The antibodies and the B cells from the blood sample are purified. The tissue sample is converted to a cell culture and then a total genome, total transcriptome and total proteome are produced from this cell culture. These arrays contain all the DNA, RNA and proteins against which the organism can develop a reactivity. Then the purified antibodies are added to these arrays. If immunogens are present at the level of DNA, RNA or protein, the antibodies will bind to them. Therefore, each spot to which the antibodies bind constitutes an autoimmunogen. By obtaining the immunogens of the copies, it is then possible to check on whether the B cells can be activated with them and whether there is thus an autoimmunity to these autoimmunogens. If these autoimmunogens have been identified, a corresponding treatment can be developed, so that the autoimmunity is diminished, delayed or even canceled. However, this method serves only to identify the autoimmunogens and not to establish a treatment.

It is also preferable to use the method according to the present invention for epitope screening for elucidation of allergies. Using this method, it is possible to ascertain whether there are already known epitopes that can cause an allergy at the level of the DNA, RNA or proteins. This method corresponds largely to the epitope screening for vaccines. The organism that has an immune system is the organism to be investigated itself which suffers from an allergy. In any case, the organism has the corresponding antibodies in its blood stream which were generated by means of an immune defense and have made the organism now reactive to the allergen. A blood sample is taken of this organism. The antibodies and the B cells from this blood sample are purified. In addition, a DNA pool that codes for known epitopes at the level of DNA, RNA or protein is created. This pool is used to create a DNA original and copies of it in the form of DNA, RNA and protein are created. Since the pool contains known allergens in coded form, the arrays consist of known allergens. Then the purified antibodies are added to these allergen arrays. If allergens are present at the level of DNA, RNA or protein, the antibodies will bind to them. Based on the position to which the antibodies can bind, a sequence and thus the triggering species can then be assigned to the species of the allergy. Then both the allergens and the species themselves may be added to the B cells to check on whether the B cells react to the presence of the species. Thus with this method it is possible to test a very large number of molecular antigens to determine whether there is an allergy and then an opposing test is performed by means of the B cells. However, this method does not detect allergens that have not previously been described or characterized.

It is also preferable to use the method for epitope screening for allergen elucidation. Using this method it is possible to determine whether there are epitopes that trigger an allergy at the level of the DNA, RNA or proteins. This method corresponds largely to epitope screening for vaccines. This requires an organism that has an immune system and of which it is known that it has developed an allergy to a certain species. A blood sample is taken of the organism and antibodies and B cells are obtained from it. Samples are taken from the species to which the allergy exists and these samples are used to create total genome arrays, total transcriptome arrays and total proteome arrays. If allergens exist on a DNA, RNA or protein level, then they will be present in the resulting arrays. The purified antibodies are then added to the arrays. If there is an allergen thereto, then the antibodies will bind to it. The sequence of the allergen can be derived on the basis of the position and sequencing. Recovered allergens are then added to the B cells. If the B cells exhibit a reaction and thus confirm the allergen, unknown allergens can then be identified with this method on the level of DNA, RNA or proteins.

Furthermore, the method is also preferably used for optimizing the binding by means of displays. This method allows optimization and derivation of a system for optimization of an interaction between a molecule and its binder. If a DNA, RNA or protein binder on a molecule is known, then this can be varied in the form of a combinatory DNA library.

This DNA pool may contain up to 10¹⁵ different molecules and is therefore restricted by means of a display method to fewer than 10⁶ binders having a high affinity. This DNA pool can then be used to create a DNA original. Corresponding copies in the form of DNA, RNA or protein are then created and the molecule is added to these arrays. Spots that have a particularly strong binder will bind a particularly large number of molecules and thus will generate a very high signal. Since all the spots of the array contain binder mutants, it is to be expected that a very high number of binders will be detected. On the basis of the sequencing of the DNA original or a DNA copy, the sequence information can then be correlated with the affinities of the binding and sequence patterns that allow a prediction of affinities when there are changes in the sequences can be derived. It is thus possible to develop possible individual binders in a targeted manner, so that they have precisely defined affinities or properties. This method thus allows optimization of the binding properties as well as a derivation of a system for predicting affinities for these binders.

Furthermore, it is preferable to use the method for binding optimization by means of a scan. This method allows optimization and derivation of a system for optimization of an interaction between a molecule and its binder. If a DNA, RNA or protein binder to a molecule is known, it can be varied in DNA in coding form systematically or randomly in one or more positions at the level of the DNA. The number of different binder mutants is kept to less than 10⁶, so that all the mutants of the DNA pool that are created can be transferred directly into a DNA original according to section 2.2.1. The DNA original is then copied to DNA, RNA or protein, depending on the binder, and the resulting binder mutation arrays are the incubated with the molecule. It may be assumed that a great many bonds will be formed. The sequences of the respective spots are assigned to the affinities and a system is derived therefrom. This procedure corresponds to the alanine scans in proteins but in this case it can be performed with any possible replacement. Thus a significantly larger number of mutations is covered and the derived system therefore has a much greater relevance.

It is also preferable to use the method for protein function screening. This method makes it possible to clarify on a molecular level by replacement of amino acids, which amino acid position is important for the functionality of the protein. If a function in the form of a binding of an activity is known of a protein, then this is varied in the form of coding DNA. Variations are then inserted systematically or randomly in one or more positions at the level of the DNA. The number of different mutations is kept to less than 10⁶, so that all the mutants of the DNA pool that are created can be transferred directly to a DNA original. The DNA original is then copied to protein. The resulting protein mutations are tested and characterized for binding or activity. It may be assumed that all the mutants exhibit binding or activity which can often vary greatly, however. The sequences of the respective spots are assigned to the respective activity of the mutants and a system is derived therefrom. This procedure corresponds to the alanine scan in proteins, but in this case it can be performed with any possible replacement. A significantly larger number of mutations is thus covered and the system therefore derived has a much greater informational value. It is thus possible to make a statement not only about which amino acid is extremely important and must not be replaced by alanine but also which substituents are possible for the previous amino acid in order to preserve the functionality of the protein.

Use for optimization of the reaction of enzymes is also preferred. This method is aimed at optimization of enzymes with regard to their conversion by adjusting the reaction conditions and cofactors. To do so, the surface of a property storage is coated completely with a single enzyme and then substrate is added. For each position, the amount of substrate created is analyzed over the entire storage. Based on the position in the storage, the optimal reaction condition can then be determined. It is thus possible to investigate up to 10⁶ different reaction conditions in a single test. This system is then preferably used for optimization of concentrations of substrate and known cofactors such as salts, substrate, pH and temperature.

As soon as the optimum reaction conditions have been found, screening can be performed using unknown cofactors and/or mutations of the cofactors. To do so, a particle transfer storage is filled with particles, each of which contains a mutant of a cofactor. These cofactors have been created as a combinatorial chemical library. Then a DNA copy, which allows decoding of the molecular structure of the cofactor on the basis of its sequence information, is created. Then the storage having the enzyme and substrate is filled under optimized conditions. Next there is a measurement of which cofactor leads to an improved conversion. This cofactor can then be determined on the basis of the sequencing. This system thus first allows optimization of the enzymatic reaction and then also the screening of previous mutations of one or more cofactors. The reaction can be optimized further in this regard.

Use for active ingredient screening by addition of the interaction partner is also preferred. This method makes it possible to discover an active ingredient for a given molecule with which the active ingredient should interact. Therefore particles of a chemical library are already provided with a molecular tag in synthesis, allowing an assignment of the molecular structure of the active ingredients synthesized on this basis to each particle after sequencing. The particles are then transferred to a particle transfer storage and a few copies are produced. The microarrays thereby created contain either the active ingredients or the coding DNA and/or RNA. Then the DNA sequence is determined by sequencing for each spot on the array and thus the molecular structure of the active ingredients is calculated. The following embodiments are now preferred:
Different interaction partners, for example, a binder is now added to the individual active ingredient arrays and the interaction is measured. Spots with an especially strong signal interact especially strongly and thus represent active ingredients having a potentially strong effect. After such a strong interaction has been identified, the active ingredient is recovered or synthesized again and the interaction is validated again using classical methods.
However, the interaction partner may also be mixed in advance with its natural interaction molecule or with the previous active ingredient. A competition then develops when this mixture is then applied to the active ingredient array. Only active ingredients having a stronger bond than the active ingredient or the natural interaction partner can generate a signal. Particularly potent active ingredients can thus be identified.

The use of a DNA tag and/or an RNA tag permits easy identification of the molecular structure on the basis of the sequencing. On particles, this is often possible only with a great effort. This method thus constitutes a great simplification. Alternative labeling methods such as mass spectrometry tags or NMR tags are also conceivable. Then a copy is created in the form of a microarray suitable for mass spectrometry or NMR. An embodiment in which the particles are labeled in the form of the chips they contain or fluorophores and can thus be assigned is also preferred. It is therefore possible with this method to investigate a large number of active ingredient variants and to assign the molecular structure easily on the basis of the DNA copy.

It is also preferable to use the method for optimization of active ingredients. This method corresponds in principle to the discovery of active ingredients as described above according to the active ingredient screening by adding the interaction partner. However, the active ingredient is already known in these cases and a combinatory chemical library is created, which has similarities for the active ingredient already identified to a very great extent. The particles of the chemical library are provided with a molecular tag according to section 2.2.8, thus allowing the molecular structure of the active ingredient synthesized thereon to be assigned to each particle after sequencing. The particles are then transferred to a particle transfer storage and a few copies are produced. The microarrays thus created carry either the active ingredients or the coding cDNA and/or RNA. Then, by means of sequencing, the DNA sequence is determined for each spot on the array and thus the molecular structure of the active ingredients is calculated. The following embodiments are now preferred:
The interaction partner, for example, a binder is added to the active ingredient array and the interaction is measured. Spots having a particularly strong signal will interact particularly strongly and thus will constitute active ingredients having a strong effect.
However, the interaction partner may also be mixed with its natural interaction molecule or the previous active ingredient. When this mixture is added to the active ingredient array, a competition takes place. Only active ingredients having a stronger binding than the active ingredient or the natural interaction partner can now generate a signal. Particularly potent active ingredients can thus be identified.

The use of a DNA tag and/or RNA tag permits simple identification of the molecular structure on the basis of the sequencing. This is often possible with the particles only with great effort. This method therefore constitutes a definite simplification. Alternative labeling methods such as mass spectrometry tags or NMR tags are also conceivable. Then a copy is created in the form of microarray suitable for mass spectrometry or NMR. An embodiment in which the particles are labeled in the form of chips or fluorophores contained therein and thus can be assigned on this basis is also preferred. It is thus possible with this method to investigate a large number of active ingredient variants and to assign the molecular structure easily on the basis of the DNA copy.

Furthermore, it is preferable for the method to be used for screening for viral points of attack. The DNA and the mRNA are obtained from a species, i.e., the host and then a total genome, total transcriptome and total proteome are created therefrom. Then the DNA, RNA and protein are obtained from a tissue sample of the host and a parasite of the host and are purified. The respective samples are then labeled with different colors, combined and each is added to the individual arrays. Since a parasite must dominate the host molecularly in some form, there must be spots where the DNA, RNA or the protein binds better (with a higher affinity) than is the case with the host itself. These spots are, so to speak, the molecular points of attack of the parasite. This is true in particular when a virus is involved. Corresponding spots on the level of the DNA, RNA or protein arrays then have a more intense coloration of the parasite. These interactions between parasite and host constitute the initial interactions with which viruses in particular can take over a cell by penetrating into it or assuming and/or replacing molecular functions. If this points of attack are known precisely, then one can search for active ingredients for precisely these interactions such that these ingredients suppress or at least interfere with the interaction between parasitic DNA, RNA or protein with the DNA, RNA or the proteins of the host. Such active ingredients can then be used as an “antiparasitic” agent. In particular for antiviral active ingredients, it is thus possible to assign the efficacy to a molecular interaction in that the interaction pairs can be identified for the first time throughout the genome, proteome and transcriptome in one batch by using this system.

It is preferable for this use to take place in a screening method for identification of antibiotics, inhibitors of antibiotics, for antibody optimization, antibody stabilization, antibody isolation, epitopes for autoimmune diseases, epitopes for allergies, epitopes for allergens, epitopes for vaccines, active ingredients, interaction partners for active ingredients, optimizations for active ingredients, growth factors, substituents for growth factors, optimization of growth factors and/or virus attack points.

It is also preferable for this to be used in a screening method for identification of molecular stability, preferably of DNases, RNases, proteins, kinases and/or phosphatases.

The advantages of the invention are manifested in particular in combination of its throughput because of the use of reaction steps, application-oriented creation of the first storage and creation of one or more similar or different copies and assignment of the analyses of individual copies and/or of the original. These advantages make it possible to elucidate the structure of the original molecules as well as their derivatives and amplificates as well as the assignment of properties of same. The spectrum of possible analyses is broadened because the reaction step, preferably the copy process can also be used for the analysis. With this invention it is possible to investigate a great many molecules in a targeted manner (10² to 10⁶ or even more), to determine their structure and to compare their similarities and differences in structure as well as properties with one another. The increased throughput improves the pre-existing screening and display methods but also allows entirely new applications.

Special advantages include the separate determination of individual or multiple properties and the structure of molecules on separate microarrays which are derived from one another by copying and thus have a “relationship” to one another. Furthermore, the correlation of these properties on the basis of the positional information on the microarray copies is such that the respective properties can be assigned to each original molecule, its amplificates and derivatives on the copies and compared with one another in a particularly advantageous manner. The optional use of the reaction and transfer step as a method of analysis in order to detect additional properties of molecules or biochemical processes entails additional special advantages.

Through the present invention it has surprisingly been possible to make available a method, which can be used for analysis of molecular properties and/or reaction conditions, wherein the method proceeds particularly rapidly and smaller volumes of reaction solutions must be used. It is thus possible to save on time and costs. Furthermore, this method makes it possible to perform an automated process, which also entails cost savings and permits an increase in efficiency.

EXAMPLES

The invention will now be illustrated on the basis of an example below without being limited thereto.

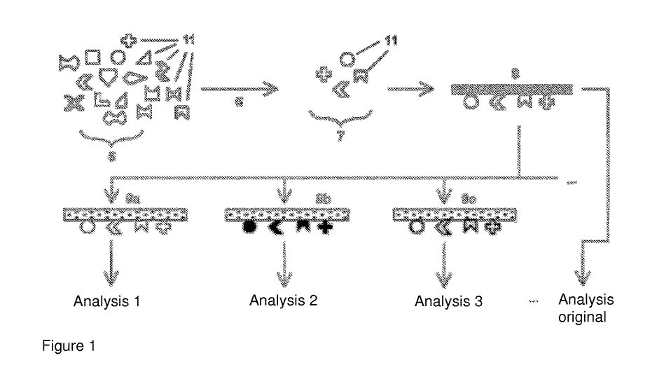

FIG. 1 shows a preferred embodiment of the invention. First, there is a starting pool 5 of sample molecules 11. If the number of molecules in this pool is significantly greater than the amount of “pixels” which can be managed in a transfer storage, then the number of molecules must be reduced. This can be accomplished through a suitable selection 6, such as that used in the display methods, for example. Then the first storage 8 is created from this copy pool 7 (step 1). The original is a spatially fixed arrangement of molecules of the copy pool. Each position on the original is clearly linked to one or more molecules. This original is “copied” in a suitable form (step 2). Various copies 9a, 9b, 9c, . . . are possible here. Next both the original and the copy and/or the copy process itself are analyzed (step 3).

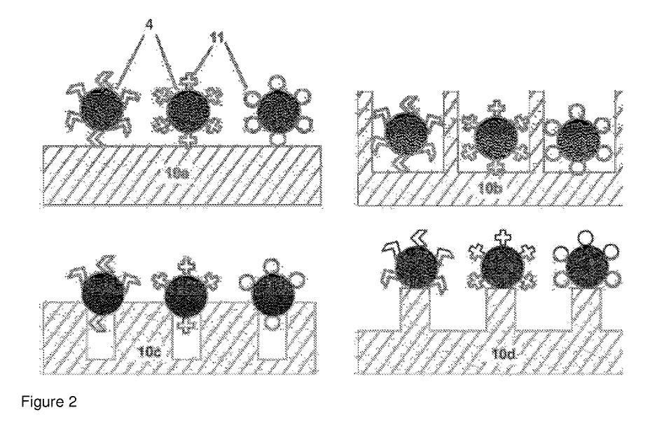

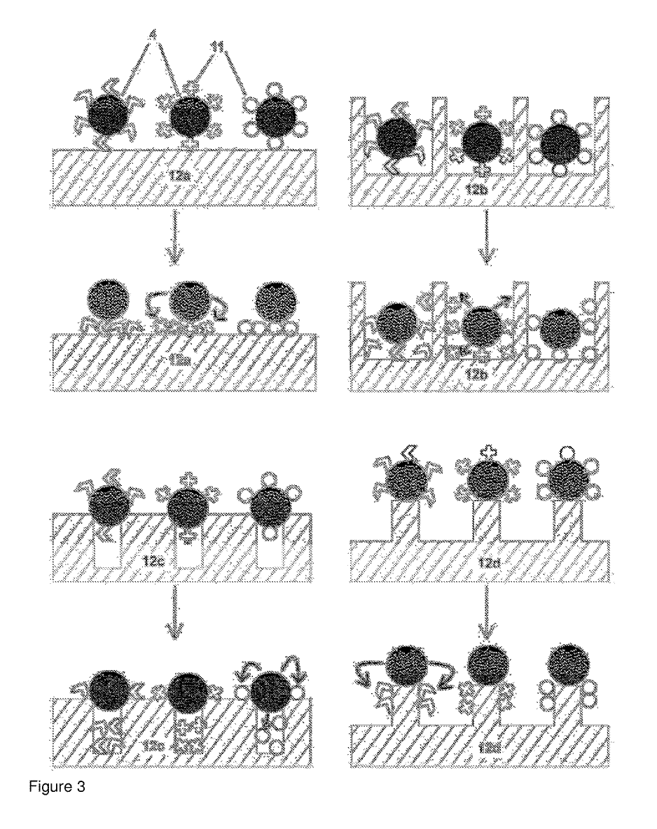

FIG. 2 shows a particle storage 10a-d as the first storage 8. Particles 4 with molecules 11 are added onto the surface and remain there. The particles may be added onto a planar surface 10a, in a structure 10b, between structures 10c or on structures 10d.

FIG. 3 shows a schematic drawing of a particle transfer storage. This illustrates how particles 9 [sic; 4] with molecules 11 can be added to the surface and remain there. The particles may be added onto a planar surface 12a, into a structure 12b, between structures 12c or onto structures 12d. Then at least one species of molecule is transferred to the surface of the storage by means of splitting, amplification or derivatization.

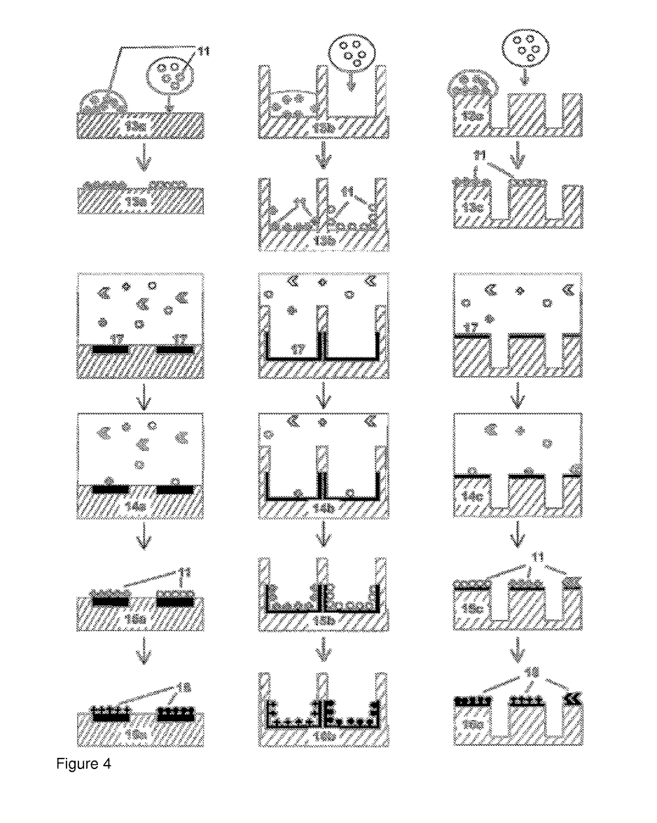

FIG. 4 shows the schematic drawing of a preferred molecular storage. Molecules 11 are added to the storage. This can take place through a dispensing process and can thus lead to a spatial arrangement of the molecules 13a-c. In addition, the molecule can be applied to the surface 14a, into structures 14b or onto structures 14c by means of a liquid contact or filling, in particular when binding regions 17 of the surfaces form preferred binding sites for the molecules. In the preferred embodiment shown here, there is one molecule in such a binding region 17. The original molecule 11 can be replicated with spatial resolution by means of a subsequent amplification 15a-c and optionally following derivatization 16a-c, in particular when the regions 17 of the surface are advantageous or essential for the amplification or derivatization. Identical molecules 11 at 13a-c, 14a-c and 15a-c or derivatives 18 at 16a-c are anchored on the surface, depending on the embodiment. Each of these embodiments 13 to 16 may then serve as a molecular storage and may release or create molecules for creating a copy in a subsequent amplification reaction and/or derivatization reaction.

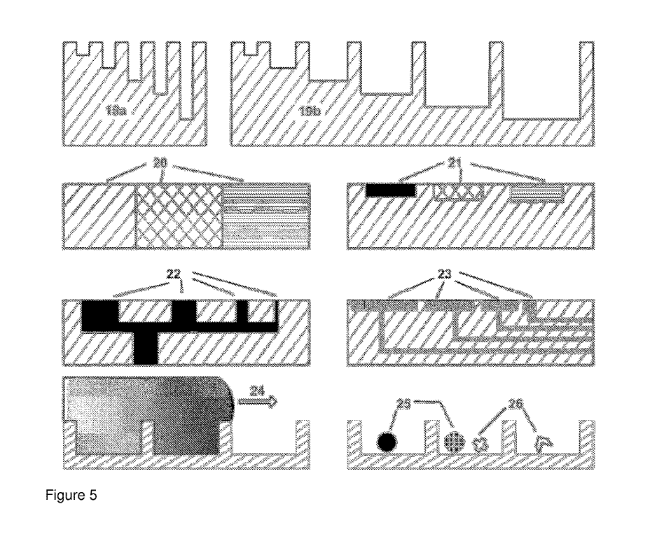

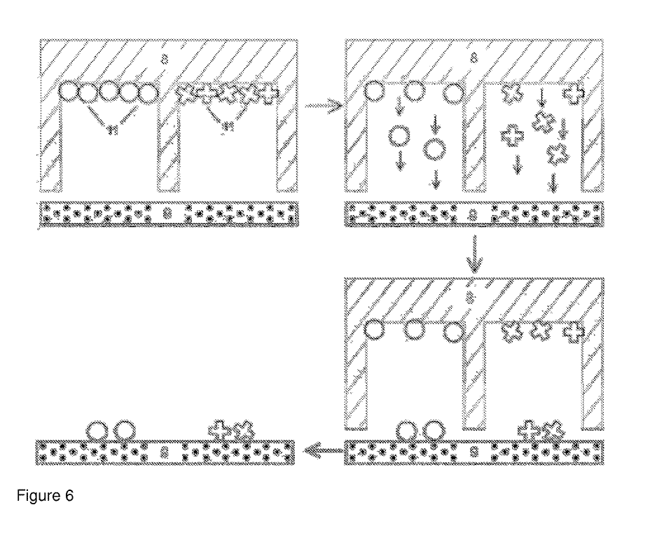

FIG. 5 shows a schematic drawing of the property storage. Each physical position of the property storage has different properties. These differences may occur due to the geometry 19a and 19b, the choice of material 20, the surface coating 21, integrated microfluidics 22 or microelectronics 23, differences in the liquid which may occur due to the filling process 24 itself or may be created due to additional particles/molecules 25/26 that are added and can alter the chemical or physical environment.

FIG. 6 shows a schematic drawing of the transfer copy. Molecules 11 are released from the original 8 and are transferred to the copy surface 9. This causes a reduction in the number of molecules in the original.

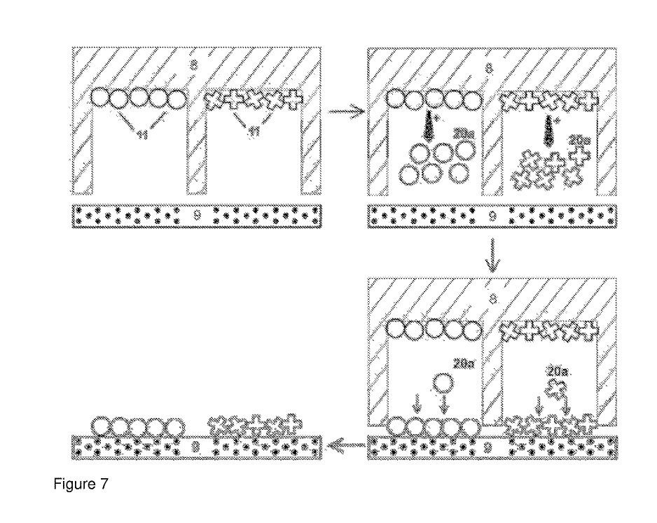

FIG. 7 shows a schematic drawing of the amplification copy. Amplificates 20a are created from the molecules 11 of the original and are then transferred to the copy.

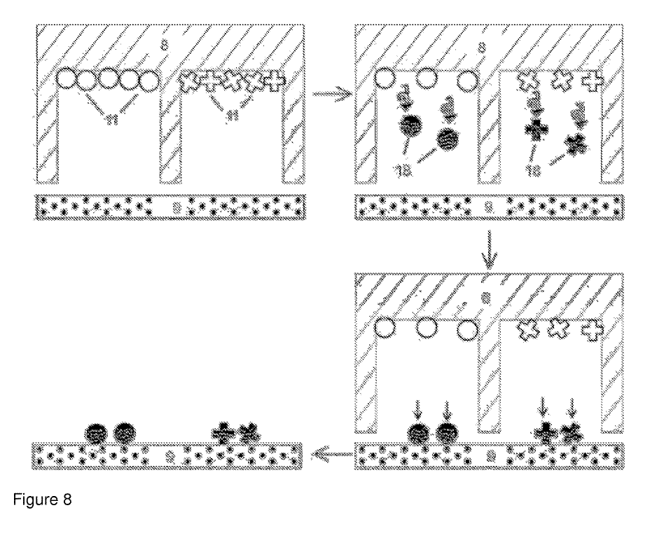

FIG. 8 shows a schematic drawing of the derivatization copy. The molecules 11 of the original are derivatized 18 and then transferred to the copy surface 9.

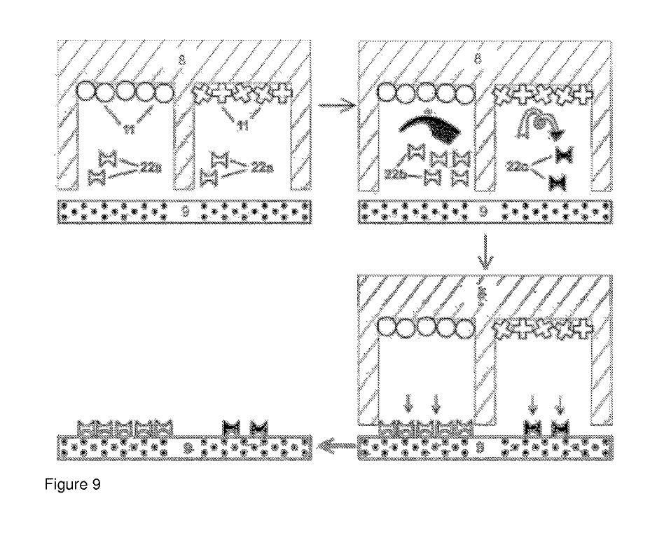

FIG. 9 shows a schematic drawing of the self-created copy. Under suitable conditions, the molecules 11 of the original have a reactivity which can be utilized to create amplificates 22b or derivatives 22c of the added molecules 22a. The molecules thereby created can then be transferred.

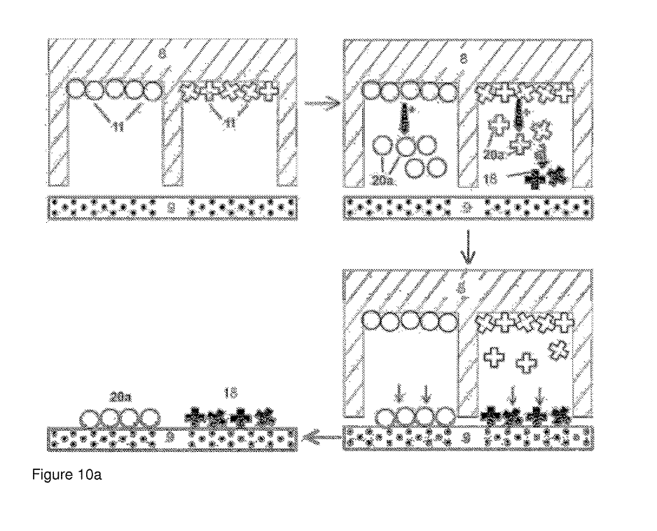

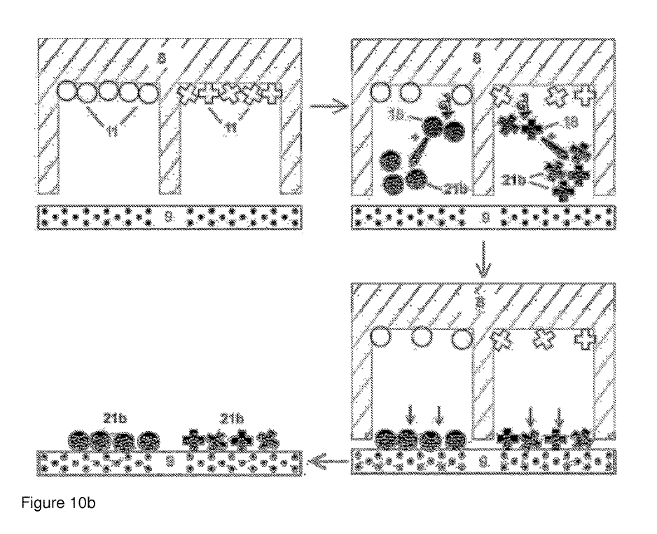

FIG. 10a shows a schematic drawing of the combination copy (preserving the original first storage). In the preferred process management, amplificates 20a are produced first and then are optionally transferred directly or are derivatized 18 and then transferred.

FIG. 10b shows a schematic drawing of the combination copy (in which the original sample molecules are used up), but derivatives 18 may also be produced first, then amplified 21b and transferred.

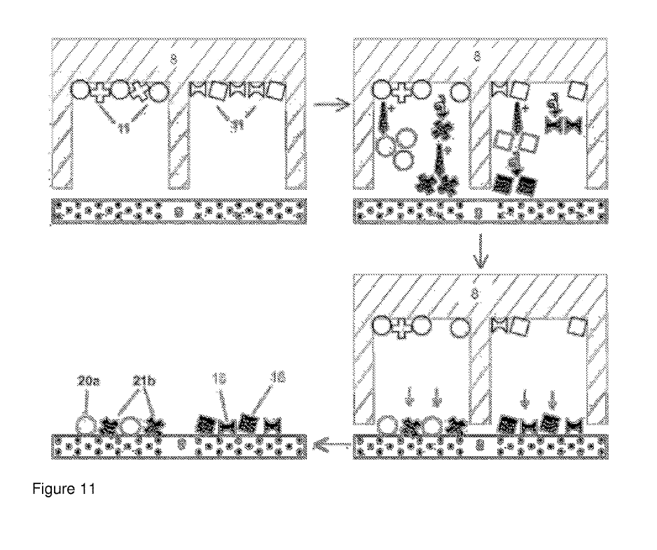

FIG. 11 shows a schematic drawing of the multi-molecule copy. Through a suitable choice of the process management, two species of molecules 11 of the original 40 are used to create a copy having at least two species of molecules derived therefrom, for example, direct amplificates 20a, derivatives 18, derivatized amplificates 18 or amplified derivatives 21b.

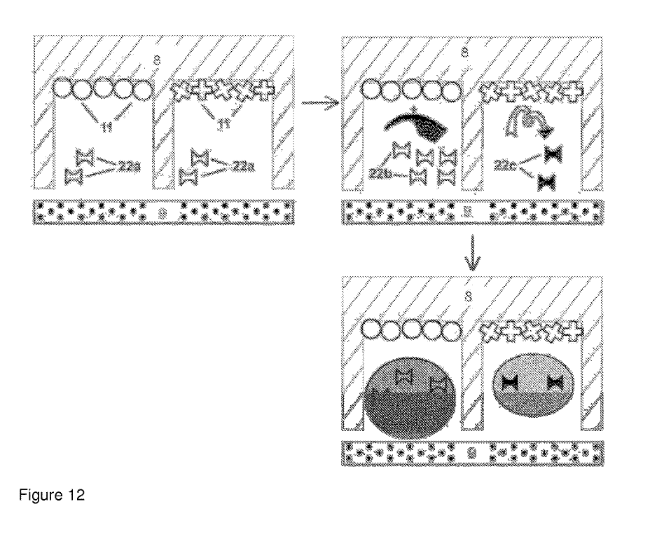

FIG. 12 shows the schematic sequence of the liquid copy. By directly adding molecules 22a that can be converted directly to the original, this forms derivatives 22c or amplificates 22b of these added molecules. In contrast with the previous embodiments, derivatives and/or amplificates that are created will remain in solution and are not transferred to a copy. This embodiment then detects the amplificates and/or derivatives thus created in solution (shown here in gray).

FIG. 13 shows the schematic sequence of the ribosome copy. First, the ribosome display is performed according to the prior art. The RNA (30) is brought in contact with ribosomes 31 and these then create the corresponding proteins 32. Then the desired target 33 is added and the ribosomes whose appended protein has bound the target are selected. This selection permits enrichment of ribosomes and/or RNA which are coupled to an interacting protein. This RNA 30a which codes for a binding protein can then be introduced into an original according to section 2.1.1, so that an RNA original 34 and/or a DNA original 35 is/are created. A preferred embodiment is a DNA original in which the DNA is amplified 36. There are microarray copies with which DNA, RNA and protein are created. The DNA copy 37 or the original itself can be sequenced, so that this yields sequence information while the RNA copy 38 is again used with ribosomes and the protein copy 39 is tested again for binding to the target. All of this again confirms the binding to the target and is used to elucidate the sequence.

FIG. 14 shows the schematic sequence of the phage copy. First, the phage display is performed according to the prior art. The phages 40 carry proteins 41 which correlate with the DNA 42 in their interior. By binding to a target 33, the phages 40a, which carry a protein that binds to the target, can be enriched by means of targeted selection. The DNA 42a which codes for a binding protein can then be introduced into an original and preferably consists of DNA 34 or amplified DNA 35. However, an RNA original 36 is also conceivable. There are microarray copies in the form of DNA, RNA and protein. The DNA copy 37 or the original itself may be sequenced, so that sequence information is obtained. The RNA copy 38 may be used for a ribosome display and a protein copy 39 may again be tested for binding to the target to thus validate the interaction with the target.

FIG. 15 shows the synthesis and use of the combinatory chemistry copy. Even during synthesis (according to FIG. 1), a DNA (or optionally an RNA) is added in parallel in each step in which a chemical building block is incorporated. Each particle 4 thus also carries DNA 52 in addition to the molecules 11. Based on the synthesis strategy, it is possible to conclude clearly from the sequence of the DNA in which of the “splits” the particle respectively was located. Then the particles of the library are analyzed for binding to a target 33 and particles having those molecules are enriched with binding molecules 51a. The resulting binding particles 50a are inserted into an original. In one preferred embodiment, this is a particle storage 10a. Then both DNA copies 37 and molecular copies can be created by derivatization 56 or amplification 57 by means of the particle storage. Based on the DNA copy, the sequence and thus the chemical structure of the molecules on the molecular copy are determined. Using the molecular copies, a binding measurement to the target can be performed again.

### LIST OF REFERENCE NUMERALS

### 4 Particle

5 Starting pool of sample molecules

### 6 Selection

7 Selected sample molecules
8 First storage
9a Transfer storage I
9b Transfer storage II
9c Transfer storage III
10a Particle storage having a planar surface
10b Particle storage having a structured surface, with particles in the structure
10c Particle storage having a structured surface, with particles between the structures
10d Particle storage having a structured surface, with particles on the structure Individual sample molecules
12a Particle transfer storage having a planar surface
12b Particle transfer storage having a structured surface, with particles in the structure
12c Particle transfer storage having a structured surface, with particles between the structures
12d Particle transfer storage having a structured surface, with particles on the structure
13a Molecular storage having a planar surface
13b Molecular storage having a structured surface, with sample molecules in the structure
13c Molecular storage having a structured surface, with sample molecules on the structure
14a Molecular storage having a planar surface and liquid
14b Molecular storage having a structured surface and liquid, with sample molecules in the structure
14c Molecular storage having a structured surface and liquid, with sample molecules on the structure
15a Molecular storage having a planar surface after amplification
15b Molecular storage having a structured surface and liquid, with sample molecules in the structure after amplification
15c Molecular storage having a structured surface and liquid, with sample molecules on the structure after amplification
16a Molecular storage having a planar surface after derivatization
16b Molecular storage having a structured surface and liquid, with sample molecules in the structure after derivatization
16c Molecular storage having a structured surface and liquid, with sample molecules on the structure after derivatization
17 Binding region on the surface

### 18 Derivatives

19a Property storage I having cavities
19b Property storage II having cavities
20 Property storage having different materials

### 20a Amplificates

21 Property storage having different surface coatings
21b Amplificate of a derivative
22 Property storage having integrated microfluidics
22a Molecules added additionally
22b Amplificates of molecules added additionally
22c Derivatives of molecules added additionally
23 Property storage having integrated microelectronics
24 Filling operation
25 Particles that change the chemical or physical environment
26 Molecules that change the chemical or physical environment

### 30 RNA

30a RNA that codes for a binding protein

### 31 Ribosome

32 Protein created

### 33 Target

34 First storage having RNA
35 First storage having DNA
36 First storage having DNA already amplified
37 Transfer storage having DNA copy
38 Transfer storage having RNA copy
39 Transfer storage having protein copy

### 40 Phages

40a Phages that carry 41
41 Protein that correlates with DNA in the interior of the phages
42 DNA in the interior of the phages
42a DNA that codes for 41
50a Particles having 51a
51a Target-binding molecule
52 Additional DNA on particle
56 Transfer storage using derivatives
57 Transfer storage using amplificates

1. A method for analysis of molecular properties and/or reaction conditions, comprising:
a) supplying a first storage, comprising a first surface, wherein
a selection of sample molecules is bound directly or indirectly to the surface in a defined arrangement,
b) of producing at least two transfer storages, wherein
at least two additional surfaces are provided, and
carrying out a reaction, the reaction being a transfer reaction, amplification reaction and/or a derivatization reaction, so that product molecules are formed, and these product molecules and/or the sample molecules bind to the surfaces, wherein
there is a clear-cut spatial association between the sample molecules of the first storage and the product molecules and/or the sample molecules of the transfer storage,
c) analyzing the first storage, the transfer storage, the sample molecules, the product molecules, the transfer reaction, the amplification reaction and/or the derivatization reaction.

2. The method according to claim 1, wherein the selection of sample molecules is made from a pool of sample molecules.

3. The method according to claim 1, wherein the selection of sample molecules is prepared via mutations and permutations of a starting molecule.

4. The method according to claim 1, wherein the sample molecules are bound to particles.

5. The method according to claim 1, wherein different species of sample molecules are bound to a particle.

6. The method according to claim 1, wherein the surface of the first storage and/or of the transfer storage is/are structured.

7. The method according to claim 1, wherein the sample molecules and/or product molecules are proteins, enzymes, aptamers, antibodies or parts thereof, receptors or parts thereof, ligands or parts thereof, nucleic acids, nucleic acid-type derivatives, transcription factors and/or parts thereof, molecules that were created by combinatory chemistry.

8. The method according to claim 1, wherein the reaction is performed via DNA polymerase, RNA polymerase and/or a cell-free reaction mixture.

9. The method according to claim 1, wherein the structuring of the surface is selected from the group comprising cavities, elevations, cavities containing particles and/or elevations surrounding particles.

10. The method according to claim 1, wherein the first storage has, in different regions, different physical, chemical and/or biochemical properties.

11. The method according to claim 1, wherein during the reaction at least one species of sample molecules is dissolved from the surface, wherein the surface optionally comprises particles.

12. The method according to claim 1, wherein the analyzing comprises a label-free method.

13. The method according to claim 1, wherein the analyzing comprises a method which uses a label.

14. The method according to claim 1, wherein the analyzing comprises a method which analyzes the solution above the surface of the first storage and/or of one of the transfer storages.

15. The method of claim 1, wherein the method screens for transcription factors, transcription efficiency, transcription optimization, promoter efficiency, spliceosomes, restriction substrates, amplification system, codon optimization, protein functionality, enzyme functionality, enzyme optimization, isoenzymes, ribozymes, reaction optimizations and/or binding optimization.

16. The method of claim 1, wherein the method screens for antibiotics, inhibitors of antibiotics, antibody optimization, antibody stabilization, antibody isolation, epitopes for autoimmune diseases, epitopes for allergies, epitopes for allergens, epitopes for vaccines, active ingredients, interaction partners for active ingredients, optimizations for active ingredients, growth factors, substituents for growth factors, optimization of growth factors and/or viral attack points.

17. The method of claim 1, wherein the method screens stability of molecules, preferably DNases, RNases, proteins, kinases and/or phosphatases.

18. the method of claim 10, wherein the different physical, chemical and/or biochemical properties are different volumes of the cavities, differences in pH, differences in salt content, temperature differences, different surfaces, differences in wettability, differences in electric charge, differences in electrical, magnetic and/or dielectric properties, differences with respect to osmotic pressures, different additives, different biochemical ingredients.

19. The method of claim 12, wherein the label-free method is an RIfS detection, iRlfS detection, Biacore detection, surface plasmon resonance detection, ellipsometry, mass spectroscopy, detection of the increase in mass, detection of the change in refractive index, detection of the change in the optical, magnetic, electrical and/or electromagnetic properties.

20. The method of claim 13, wherein the method using is label comprises a fluorescence measurement, detection via an absorbent and/or scattering dye, mass spectroscopy via detection of an isotope label, detection via a molecule which changes the refractive index and/or the optical properties of the surface and/or of the solution.
